(19) United States
(12) Patent Application Publication
(10) Pub. No.: US 2024/0342296 A1
(43) Pub. Date: Oct. 17, 2024

(54) pH LOW INSERTION PEPTIDE TARGETED DELIVERY OF POTENT CYTOTOXIC COMPOUNDS

(71) Applicants: University of Rhode Island Board of Trustees, Kingston, RI (US); YALE UNIVERSITY, New Haven, CT (US)

(72) Inventors: Yana K. RESHETNYAK, Saunderstown, RI (US); Oleg A. ANDREEV, Saunderstown, RI (US); Anna MOSHNIKOVA, Warwick, RI (US); Donald M. ENGELMAN, New Haven, CT (US)

(21) Appl. No.: 18/608,699

(22) Filed: Mar. 18, 2024

Related U.S. Application Data
(63) Continuation of application No. 16/774,966, filed on Jan. 28, 2020, now Pat. No. 12,029,793.
(60) Provisional application No. 62/797,881, filed on Jan. 28, 2019.

(51) Int. Cl. A61K 47/64; A61K 9/00; A61K 31/4745; A61K 31/5386; A61K 31/7036; A61K 38/12; A61K 47/60; A61P 35/00

(52) U.S. Cl. CPC A61K 47/6425; A61K 9/0014; A61K 9/0034; A61K 31/4745; A61K 31/5386; A61K 31/7036; A61K 38/12; A61K 47/60; A61K 47/6415; A61P 35/00

(57) ABSTRACT

The invention features a composition comprising a potent cytotoxic compound and a pHILIP® peptide, where, e.g., the cytotoxic compound cannot be used alone due to a lack of targeting. pHILIP® peptide targets cytotoxic compounds to acidic diseased tissue, translocates cytotoxic compounds across plasma membranes into the cytosols of cells in acidic diseased tissues and induces cell death predominantly in the targeted acidic diseased tissue.

### RELATED APPLICATIONS

This application is a continuation of 16/774,966, filed on Jan. 28, 2020, which claims the benefit of priority under 35 U.S.C. § 119(e) to U.S. Provisional Application No. 62/797,881, filed Jan. 28, 2019, the entire contents of which are incorporated herein by reference in its entirety.

### STATEMENT AS TO FEDERALLY SPONSORED RESEARCH

This invention was made with government support under R01 GM073857 awarded by the National Institute of General Medical Sciences of the National Institutes of Health. The government has certain rights in the invention.

### INCORPORATION BY REFERENCE OF SEQUENCE LISTING

The contents of the sequence listing text file named “5095_0110003_SequenceListing_ST26.xml”, which was created on Mar. 15, 2024 and is 326,361 bytes in size, is hereby incorporated by reference in its entirety.

### FIELD OF THE INVENTION

The present invention relates to chemotherapy.

### BACKGROUND

Conventional methods of cancer treatment, such as non-targeted chemotherapy, are toxic and do not distinguish between normal and tumor tissues very well, and are therefore limited in their use by harmful side effects for patients. In contrast to conventional chemotherapy, targeted therapies are more efficacious and safer, and have gained increasing interest in recent years. The targeted delivery of potent cytotoxic drugs to diseased cells promises to maximize their therapeutic effects while reducing side effects. The main goal of targeted therapy is to deliver cytotoxic molecules to cells in diseased tissue while avoiding significant delivery to normal tissue.

### SUMMARY OF THE INVENTION

The invention provides a solution to the problem of specific targeting of and intracellular delivery of potent cytotoxic compounds to cells in acidic diseased tissues, to induce cell death predominantly in the targeted tissue while sparing healthy cells.

Extracellular acidosis is ubiquitous in tumors, including both primary tumors and metastases, as a consequence of their rapid metabolism. Tumor cells stabilize their cytoplasmic pH by exporting the acidity to the extracellular environment. As a result of the acidic flux and the membrane potential, the extracellular pH is lowest at the surfaces of cancer cells, where it is significantly lower than either the normal physiological tissue pH or the bulk extracellular pH in tumors. The low pH region persists at the cancer cell surface even in well-perfused tumor areas. The acidity on the surfaces of cancer cells is a targetable characteristic that is not subject to clonal selection, and the level of acidity is a predictor of tumor invasion and aggression, since more rapidly growing tumor cells are more acidic. A pH Low Insertion Peptide (pHLIP®) is a water-soluble membrane peptide that interacts weakly with a cell membrane at neutral pH, without insertion into the lipid bilayer; however, at slightly acidic pH (<7.0), pHLIP®inserts into the cell membrane and forms a stable transmembrane helix. By binding a pHLIP®, or pHLIP® equivalent, to a cytotoxic compound, it is possible to specifically target and deliver the cytotoxic compound directly to the cancerous cells and into their cytosols, due to their acidic cell surfaces.

Delivering potent cytotoxic compounds using pHLIP® peptides therefore allows selective targeting of a diseased tissue (e.g. a tumor) to increase treatment efficacy. A significant advantage of this approach is that the targeted delivery of cytotoxic compounds mediated by the pHLIP® constructs described herein is associated with few side effects for the patient, which is the main problem in the use of potent cytotoxic compounds. As a result, the used of the pHLIP® constructs described herein is associated with an increase of the therapeutic index and an increase of the therapeutic window.

Accordingly, the invention features a composition comprising a potent cytotoxic compound and a pHLIP® peptide, e.g., where the cytotoxic compound comprises a small molecule that induces cell death. The cytotoxic compound is typically 2,000 Dalton in mass or less. The potent cytotoxic compound cannot be used alone, since it can induce significant toxicity in normal tissues, possibly leading to life-threatening side effects, e.g., as defined to be Adverse Events of Grade 3-5 according to the NCI guideline of Common Terminology Criteria for Adverse Events (CTCAE) v5.0, Nov. 27, 2017. Such potent cytotoxic compounds can be only used as part of targeted therapy. Examples of potent cytotoxic compounds can be selected from the classes of tubulin inhibitors, RNA polymerase inhibitors and DNA damaging agents. Examples of cytotoxic tubulin binding compounds include maytansines, chemical derivatives of maytansine (maytansinoids) and analogs of maytansine. These compounds bind to the tubulin maytansine binding site and destabilize microtubule assembly.

Cytotoxic compounds are compounds that inhibit cell growth or promote cell death when proximate to or absorbed by a cell, and moreover, when delivered to a cell (either to the interior of a target cell or to the cell surface), are capable of killing the cell or otherwise inhibiting the proliferation of the cell. Potent cytotoxic compounds are so lethal such that they cannot be administered alone. For example, in chemotherapy as a treatment of cancer often relies on the ability of cytotoxic agents to kill or damage cells which are reproducing; which preferentially targets rapidly dividing cancer cells. For example, potent cytotoxic compounds kill normal cells at a clinically unacceptable level and thus must be specifically delivered to cancer cells (or other undesirable cells), e.g., via a highly specific cancer targeting tool such as the pHLIP® peptides described herein. Thus, a significant advantage of the compositions and methods described herein is that the use of pHLIP® peptides renders previously clinically unacceptable drugs now acceptable for safe and efficacious clinical use.

Examples of cytotoxic RNA polymerase binding compounds include amatoxins, including alpha-amanitin and chemical derivatives and analogs of alpha-amanitin, which bind RNA polymerase and stop protein synthesis.

Exemplary cytotoxic DNA-damaging compounds include: i) enediyne antitumor antibiotics including calicheamicin compounds and chemical derivatives and analogs of calicheamicin, which bind in the DNA minor groove and cause strand scission; and ii) topoisomerase I inhibitor compounds, including camptothecin and its structural analog, exatecanand other chemical derivatives and analogs of exatecan, which bind and stabilize the complex of topoisomerase I and DNA, resulting in DNA damage.

The invention provides a solution to the problem, because pHLIP® peptide sequences mediate the targeting of tumor cell acidity and subsequent specific delivery of the potent cytotoxic compound into cells in a diseased tissue. The delivery is based on direct translocation of the compound across the plasma membrane (bypassing endocytotic uptake) into the cytoplasm of a targeted cell, while exhibiting little or no translocation into a normal cell. Thus, exploiting the pHLIP® peptide to allow the use of potent cytotoxic compounds as targeted highly specific chemotherapy agents.

As used herein, the term “Drug” includes a cytotoxic compound, e.g., a potent cytotoxic compound. For example, the potent cytotoxic compound cannot be used alone, since it can induce significant toxicity in normal tissues, possibly leading to life-threatening side effects, e.g., as defined to be Adverse Events of Grade 3-5 according to the NCI guideline of Common Terminology Criteria for Adverse Events (CTCAE) v5.0, Nov. 27, 2017, hereby incorporated by reference. Other references regarding potency of cytotoxic compound and methods of evaluating potency include Kummar, S. et al. Br J Clin Pharmacol (2006) 62(1); pages 15-26 and Florento L. et al Int J Biomed Sci. (2012) 8(1); pages 76-80, incorporated herein by reference in their entireties. Such potent cytotoxic compounds can be only used as part of targeted therapy.

In some embodiments, the composition further comprises a linker between the cytotoxic compound (Drug) and the pHLIP® peptide. Exemplary linkers include a disulfide bond or an acid-labile bond. In some examples, the linker is cleavable. Exemplary cleavable linkers include those that are self-immolating. Self-immolative elimination is a spontaneous and irreversible disassembly of a multicomponent compound into its constituent fragments through a cascade of electronic elimination processes. Self-immolative elimination is driven by an increase in entropy coupled with the irreversible formation of thermodynamically stable products (e.g. CO₂). Such linkers have an advantage in that the cargo/therapeutic agent (drug) can be released in an unmodified form if it has an appropriate —NH₂ or —OH group, depicted in the schematic below:

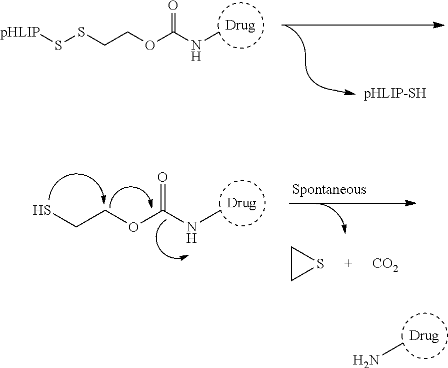

Such linkers have an advantage in that a potent cargo/therapeutic agent is released in an unmodified form. Examples also include linkers with a disulfide bond that is cleaved after delivery.

A modulator of polarity is optionally included in the composition. Such a modulator changes the overall polarity of the construct to optimize delivery to tumor cells or a tumor mass. For example, if the cargo renders the composition too polar, a modulator agent is added to make the overall composition less polar, or if the cargo is not polar enough, a modulator is added to make the composition more polar. For example, linkers comprising such modulators have an advantage in enhancing the efficiency of drug delivery into the cytosol or improving the targeting of tumors relative to normal tissues. In some examples, the construct may include a polar modulator; in other examples (such as in the case of a polar drug), the construct may include a more hydrophobic modulator to promote delivery into the cell. For example, when the cargo is polar (LogP<−0.4), the hydrophobic modulator will increase the LogP of [cargo-modulator] (LogP>−0.4). If cargo is hydrophobic LogP>2.5, the polar modulator will decrease LogP of [cargo-modulator] (LogP<2.5). Non-limiting examples of modulators are fatty acids, PEG polymers, hydrophobic fluorescent dyes, cyclic peptides.

In some examples, the composition comprises 2 or more pHLIP® peptides. Exemplary constructs comprise the following structure: Peptide—Link—B, in which Wherein “Peptide” is a first pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2), “B” is a second pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2), wherein upper case “X” indicates any amino acid residue and can include lysine (Lys), Cysteine (Cys), or an Azido-containing amino acid; and “Link” is a polyethylene glycol linker, and each “—” is a covalent bond.

Also within the invention is a method of tumor treatment comprising administering to a subject a composition comprising a potent cytotoxic compound and a pHLIP® peptide as described above. In some examples, the potent cytotoxic compound is a compound that disrupts microtubule function and induces cell death. In some examples, the potent cytotoxic compound is a compound that causes DNA strand scission and induces cell death. In some examples, the potent cytotoxic compound is a compound that binds RNA polymerase, stops protein synthesis and induces cell death. For example, the subject comprises a tumor.

The composition is administered using methods well known in the art; e.g., the composition is injected directly into a tumor mass, or administered locally by intravesical instillation, or by topical application, or the composition is systemically administered. Because of the targeting properties of the pHLIP® construct, the cytotoxic compound is specifically targeted to tumor cells and delivered into their cytoplasms.

Certain implementations comprise a formulation for a parenteral, a local, or a systemic administration comprising a pHLIP®-linker-Drug, as disclosed herein. Formulations comprising a pHLIP®-linker-Drug for intravenous, intraarterial, intraperitoneal, intracerebral, intracerebroventricular, intrathecal, intracardiac, intracavernous, intraosseous, intraocular, subcutaneous, or intravitreal administration are also provided.

In an aspect, provided herein is a formulation comprising a pHLIP®-linker-Drug for intramuscular, intradermal, transdermal, transmucosal, intralesional, subcutaneous, topical, epicutaneous, extra-amniotic, intravaginal, intravesical, nasal, or oral administration. The present subject matter also includes a formulation for intravesical instillation comprising a pHLIP®-linker-Drug as disclosed herein. In some embodiments, the formulation is used for the treatment of cancer (e.g., solid tumors) or other acidic diseased tissues.

Also provided herein is a formulation comprising a pHLIP®-linker-Drug that comprises multiple pHLIP® peptides for systemic administration. In certain embodiments, the formulation is used for the treatment of cancer or inflammation or atherosclerosis or targeting of senescent cells.

Provided herein is a method of treating cancer or inflammation or atherosclerosis or targeting of senescent cells in a subject, comprising administering to the subject an effective amount of a pH-triggered compound, wherein the compound comprises a cytotoxic compound. Non-limiting examples of cancer include bladder cancer, colon cancer, prostate cancer, breast cancer, lung cancer, skin cancer, liver cancer, bone cancer, ovarian cancer, stomach cancer, pancreatic cancer, testicular cancer, and brain cancer. In some embodiments, the cancer is bladder cancer.

Also included herein are methods for detecting and/or imaging diseased tissue (such as cancer tissue) in a subject comprising administering to the subject with a pHLIP®-Linker-Drug conjugated with imaging agent (I.A.), such as I.A.-pHLIP®-Linker-Drug.

Because of the presence of pHLIP® in the composition, the cytotoxic compound is delivered predominantly to acidic diseased tissue to induce a biological effect, cell death, predominantly in the targeted tissue. For example, the therapeutic index of the cytotoxic compound delivered in the presence of pHLIP®, e.g., in a composition that comprises both components (e.g., the pHLIP® peptide and cytotoxic compound), is at least 10%, 20%, 50%, 2-fold, 5-fold, or greater than the therapeutic index of the cytotoxic compound delivered in the absence of pHLIP® in the composition.

The composition targets the potent cytotoxic compound preferentially to a diseased tissue compared to a healthy tissue, thereby minimizing damage to the healthy tissue. In the absence of pHLIP®, the potent cytotoxic compound induces undesirable and life threatening side effects, including some or all of the following: pulmonary toxicity, neurotoxicity, ototoxicity, nephrotoxicity, hepatotoxicity, tachycardia, myelosuppression, deep vein thrombosis, oral mucositis, dysgeusia, anorexia, nausea, vomiting, diarrhea or constipation, abdominal pain, cognitive dysfunction, anxiety, depression, muscle fatigue and others. Conversely, in the presence of pHLIP®, these side effects are not observed.

Included herein are pharmaceutical compositions comprising a pH-triggered compound and a pharmaceutically acceptable carrier.

As used herein, “effective” when referring to an amount of a compound refers to the quantity of the compound that is sufficient to yield a desired response without undue adverse side effects commensurate with a reasonable benefit/risk ratio when used in the manner of this disclosure.

In some embodiments, a subject is a mammal. In certain embodiments, the mammal is a rodent (e.g., a mouse or a rat), a primate (e.g., a chimpanzee, a gorilla, a monkey, a gibbon, a baboon), a cow, a camel, a dog, a cat, a horse, a llama, a sheep, or a goat. In preferred embodiments, the subject is a human.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram of a pHLIP ® construct with a drug that is a cytotoxic compound.

FIGS. 2A-C are diagrams of pHLIP® constructs with a drug and a modulator (M) molecule. The modulator could be attached to pHLIP® peptide (FIG. 2A), linker (FIG. 2B) or Drug (FIG. 2C).

FIG. 3 is a diagram of a pHLIP® construct with 2 (or more) drugs. The drugs can be the same or different.

FIGS. 4A-C are diagrams of pHLIP® constructs with 2 (or more) drugs and a M molecule. The drugs can be the same or different. The modulator could be attached to pHLIP® peptide (FIG. 4A), linker (FIG. 4B) or Drug (FIG. 4C).

FIG. 5 is a diagram of a pHLIP construct with a drug and an imaging agent (I) at the membrane non-inserting end.

FIGS. 6A-C are diagrams of pHLIP® constructs with a drug and a M molecule and an imaging agent at the membrane non-inserting end. The modulator could be attached to pHLIP® peptide (FIG. 6A), linker (FIG. 6B) or Drug (FIG. 6C).

FIG. 7 is a diagram of a pHLIP® construct with multiple drugs and an imaging agent at the membrane non-inserting end. The drugs can be the same or different.

FIGS. 8A-C are diagrams of pHLIP® constructs with multiple drugs and M molecules and an imaging agent at the membrane non-inserting end. The drugs can be the same or different. The modulator could be attached to pHLIP® peptide (FIG. 8A), linker (FIG. 8B) or Drug (FIG. 8C).

FIG. 9 is a diagram of two or more pHLIP® peptides connected to each other by PEG polymer, (or any other polymer shown by purple color) with drugs linked together via a linker molecule. The drugs can be the same or different.

FIGS. 10A-C are diagrams of exemplary pHLIP® constructs with two or more pHLIP® peptides connected to each other by PEG polymer (or any other polymer, shown by purple color) with drugs linked together via a linker molecule. The drugs can be the same or different. The modulator could be attached to pHLIP® peptide (FIG. 10A), linker (FIG. 10B) or Drug (FIG. 10C).

FIG. 11 is a diagram of an exemplary pHLIP® construct with two or more pHLIP® peptides connected to each other by PEG polymer (or any other polymer, shown by purple color) with drugs linked together via a linker molecule. The drugs can be the same or different.

FIGS. 12A-C are diagrams of exemplary pHLIP® constructs with two or more pHLIP® peptides with drugs linked together via a linker molecule. The drugs can be the same or different. The modulator could be attached to pHLIP® peptide (FIG. 12A), linker (FIG. 12B) or Drug (FIG. 12C).

FIG. 13 is a structure of an exemplary heterobifunctional self-immolating linker useful to make pHLIP® constructs containing a drug (a therapeutic cargo).

FIG. 14A is a structure of an exemplary self-immolating linker with Drug for S—S bond exchange with Cys residue of pHLIP® peptide.

FIG. 14B is a structure of an exemplary self-immolating linker with Drug for conjugation with Lys residue at pHLIP® peptide.

FIG. 15 is a chemical structure of maytansinoid, mertansine (DM1; CAS No.: 139504-50-0), where R=CH₂CH₂SH.

FIG. 16 is the chemical structure of alpha-amanitin.

FIG. 17 is the chemical structure of the enediyne antibiotic, calicheamicin

FIG. 18 is the chemical structure of a camptothecin chemical analog, exatecan (DX-8951f).

FIG. 19A is the chemical structure of pHLIP-S—S-mertansine construct.

FIG. 19B depicts images of breast MDA-MB-231 tumors collected after intratumoral (IT) and intraperitoneal (IP) treatment with pHLIP-S—S-mertansine (see FIG. 21A) compared to the control group, which did not receive any treatment. The position indicated by “—” in IT treatment group indicates the tumor disappeared in the course of treatment and could not be collected.

FIG. 19C depicts a box plot presenting mean (filled square), median, 25 and 75 percentiles (box itself) and standard deviation values of the tumor weights collected after IP and IT treatment with pHLIP-S—S-mertansine compared to the control (non-treated) group. P-level was calculated using two-tailed test.

FIG. 20A is a bar graph depicting that tumor mass reduction was tested on mice bearing murine JC and 4T1 breast cancers after multiple IP administration of pHLIP-S—S-amanitin. P-level was calculated using two-tailed test.

FIG. 20B is a line graph depicting cell viability after treatment with increasing concentrations of pHLIP-S—S-amanitin (in logarithmic scale) at pH 7.4 and pH 6.0.

FIG. 21A depicts an image of a chemical structure of calicheamicin modified with succinimidyl 3-(2-pyridyldithio)propionate (SPDP) for direct conjugation with pHLIP containing a single Cys residue.

FIG. 21B depicts images of cervical HeLa tumors collected after IP treatment with pHLIP-S—S-calicheamicin compared to the control group, which did not receive any treatment.

FIG. 21C depicts box plot presenting mean (filled squares), median, 25 and 75 percentiles (box) and standard deviation values of tumor weights after IP and IT treatment with pHLIP-S—S-calicheamicin compared to the control (non-treated) group. P-level was calculated using two-tailed test.

FIG. 22 depicts an image of a chemical structure of exatecan modified with SPDP for direct conjugation with pHLIP® containing single Cys residue.

### DETAILED DESCRIPTION

The invention provides compositions comprising of peptides with a higher affinity to membrane lipid bilayers at low pH than at normal pH, conjugated with cytotoxic drugs, to enable targeting of acidic tissue cells and delivery of the drugs into the cells by transmembrane insertion. Current drugs cannot distinguish very well between diseased and healthy tissue, thus affecting both, leading to life threatening side effects and a limited efficacy of treatment. Some compounds in current use, such as Doxorubicin, actually target normal tissues more than tumor tissues. In particular, potent cytotoxic compounds cannot be used alone due to their life-threatening side effects. However, such cytotoxic compounds might be used as a part of targeted therapy.

The compositions and methods are used to target tumors using pHLIP® to specifically deliver cytotoxic compounds into cells in diseased tissue (cancer) and thus to promote cell death predominantly within the targeted tissue. The invention provides 3 major features: i) targeting of cytotoxic compounds to tumors to induce cell killing predominantly within tumors; ii) sparing of normal cells in healthy organs and tissues to reduce the life threatening side effects associated with the use of the cytotoxic compounds alone; and iii) direct delivery of the cytotoxic compounds across cell membranes in diseased tissues, thus avoiding endosomal uptake, which would require endosomal escape of the cytotoxic compound for it to have its effect.

### Advantages of pHLIP® Targeted Delivery of Potent Cytotoxic Compounds

Various protein biomarkers overexpressed at the surfaces of cells in diseased tissue could be targeted, thus increasing efficacy of the treatment. Although many biomarkers exist that can be exploited to improve tumor targeting and treatment outcomes, such as various receptors overexpressed at the surfaces of some cancer cells, nevertheless, useful markers are not present in all tumors. Further, the heterogeneity of the cancer cell population in an individual tumor and between tumors of various patients limits the effective use of biomarker targeting technologies. In addition, rapid mutation increases the likelihood of the selection of cancer cell phenotypes that do not express high levels of the targeted biomarker. Biomarker targeting can act as a selection method that leads to the development of drug resistance and poor patient outcomes.

Thus, a current challenge in the field of targeted chemotherapy is to search for alternative, more reliable biomarkers for tumor cell targeting. Also important are methods of intracellular delivery of potent cytotoxic drugs. If targeting could be coupled with delivery, a very important advance would be in hand. The compositions and methods described herein accomplish these objectives and thus represent significant advantages over previous approaches.

### Tubulin Inhibitors

Tubulin inhibitors are drugs that interfere directly with the tubulin system. Tubulin is the major building block of microtubules, which are present in almost all eukaryotic cells, and is comprised of α-tubulin and β-tubulin subunits. Dynamic assembly and disassembly of microtubules is involved in many cellular processes such as cell structure maintenance, cell division, and intracellular transport. Disruption of microtubules induces cell cycle arrest in the G2/M phase, which makes microtubules an attractive target for drug discovery. Most tubulin inhibitors used in the clinic are natural products and their synthetic derivatives. Microtubule/tubulin inhibitors include agents promoting tubulin polymerization and stabilizing microtubule structures to block the dynamic assembly/disassembly needed for function (e.g., paclitaxel, epothilones, discodermolide and taccalonolides), and other agents inhibiting tubulin polymerization and destabilizing microtubule structures so that the microtubules cannot form (such as maytansinoids, auristatins, vinblastine and vincristine). These inhibitors are further separated into six distinct classes based on their tubulin binding sites: maytansine, vinca, taxane, colchicine, pironetin, and laulimalide/peloruside binding sites. Molecules that bind to the taxane and laulimalide/peloruside site stabilize microtubules, while compounds targeting the maytansine, colchicine, vinblastine or pironetin site destabilize microtubules.

### RNA Polymerase Inhibitors

Inhibitors of RNA polymerases prevent transcription of genetic information from DNA into RNA, which stops protein synthesis, ribozyme production and the regulatory functions of RNA, leading to cell death. Some compounds (e.g., (cytarabine or cytosine arabinoside) have multiple mechanism of actions including binding to DNA and RNA polymerases and DNA. Some other compounds including rifamycins (e.g., rifampicin (or rifampin), rifabutin, rifapentine, rifalazil, and rifaximin) and amanita toxins (e.g., α-amanitin, β-amanitin, γ-amanitin, ε-amanitin, amanullin, amanullinic acid, amaninamide, amanin, and proamanullin) have a high selectivity for RNA polymerases.

### DNA-Damaging Agents

The known DNA damaging agents include compounds that directly modify DNA bases, intercalate between bases, or form crosslinks in DNA. The DNA-alkylating agents including nitrogen mustards (e.g., cyclophosphamide, chlorambucil, and melphalan), nitrosoureas (e.g., carmustine, lomustine, and semustine) and triazenes (e.g., dacarbazine and temozolomide), alkylating-like platinum agents (e.g., cisplatin) and platinum-based analogs (e.g., carboplatin, and oxaliplatin) are used in the clinic. Antimetabolites including pyrimidine analogs (e.q., 5-fluorouracil, capecitabine, floxuridine, and gemcitabine) and purine analogs (e.g., 6-mercaptopurine, 8-azaguanine, fludarabine, and cladribine) mimic normal cellular molecules and interfere with DNA replication. Cytotoxic DNA-damaging antibiotics include anthracyclines (e.g., doxorubicin, daunorubicin, epirubicin, idarubicin, pirarubicin, aclarubicin, and mitoxantrone), bleomycins, mitomycins, actinomycin, and of enediyne antibiotics (e.g., calicheamicin and analogs), which either insert between base pairs on the two strands of DNA (DNA intercalation), or/and cause strand scission, or/and generate highly reactive free radicals, or/and induce DNA alkylation. Another class of DNA-damaging agents is topoisomerase I inhibitors (e.g., camptothecins and analogs), which bind topoisomerase I and stabilize the complex of topoisomerase I and DNA. As a result, DNA re-ligation is prevented, which leads to DNA damage.

### Targeted Therapy and Targeted Delivery of Potent Cytotoxic Compounds

A significant proportion of cancer patients suffer from acquired resistance or relapse after long-term chemotherapy treatment. Further, non-targeted drugs have nonspecific toxicity which damages normal tissues, causing serious side effects and limiting their efficacy. In contrast to conventional chemotherapy, targeted therapy is designed to affect predominantly diseased tissue, but to spare healthy cells. The main categories of current targeted cancer therapy are small molecules (including serine/threonine kinase inhibitors and tyrosine-kinase inhibitors), monoclonal antibodies (mAbs), and antibody-drug conjugates (ADCs). In addition, various nanotechnology approaches for targeted therapy are under development.

Small-molecule or mAb targeted therapy alone often shows inadequate therapeutic activity due to its low cytotoxicity and poor penetration into solid tumors. An improved approach is to use ADCs, where a mAb is conjugated with a highly potent cytotoxic drug (or payload) through an appropriate linker. An ADC is designed as targeted therapy, which targets and kills only cancer cells while sparing healthy cells. The development of ADCs faces a number of challenges: i) the antibodies used in ADCs can elicit immunogenic responses and cause fast clearance from the circulation; ii) the cytotoxic drug can be insufficiently potent, and further conjugation might lead to decreased potency; iii) the insufficient presentation of targeted antigens at the surface of a diseased cell can lead to reduced potency or failure of an ADC; iv) poor tumor penetration of antibodies; v) the endocytotic uptake of an antibody requires endosomal escape of a drug to find its cellular target; vi) for many tumors, usable surface epitopes have not been found, and vii) tumor heterogeneity can lead to clonal selection of resistant cells, leading to a resumption of tumor growth.

Among potent cytotoxic compounds, Maytansinoids, their derivatives and analogs are exemplary of tubulin inhibitors, Amanitin, its derivatives and analogs are exemplary of RNA polymerase inhibitors, and calicheamicins and camptothecins, their derivatives and analogs are exemplary of DNA-damaging agents. Each of these and other potent cytotoxic compounds needs to be targeted to a tumor and delivered into cells—a task that is achieved by using pHLIP® technology.

### pHLIP® Constructs

The invention provides compositions and methods to target tumors with pHLIP® to specifically deliver potent cytotoxic compounds to the acidic diseased tissue (such as tumors), bypass endocytotic uptake and deliver cytotoxic compounds into the cytoplasm of cells in targeted diseased tissue, and thus promote cell killing specifically within the targeted tissue only (or predominantly). As described above, a potent cytotoxic compound must be targeted to the diseased tissue otherwise side effects will preclude its clinical use.

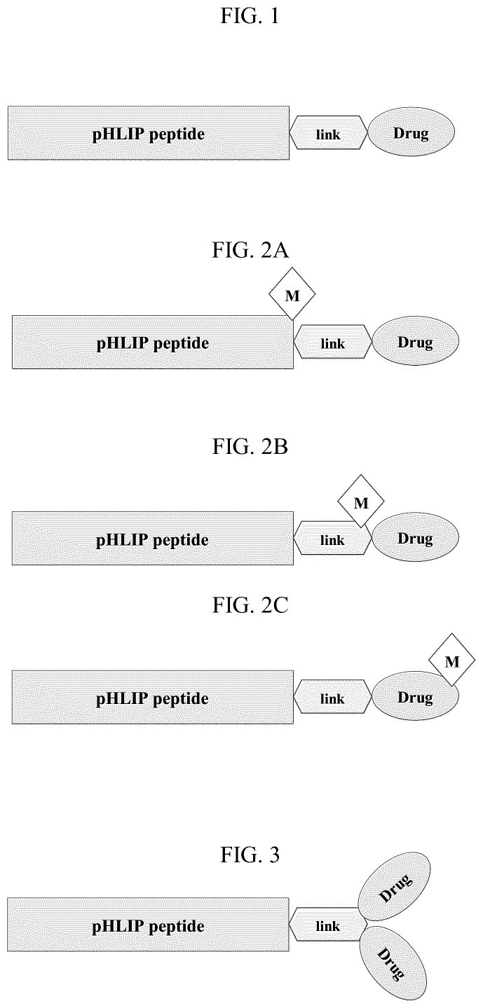

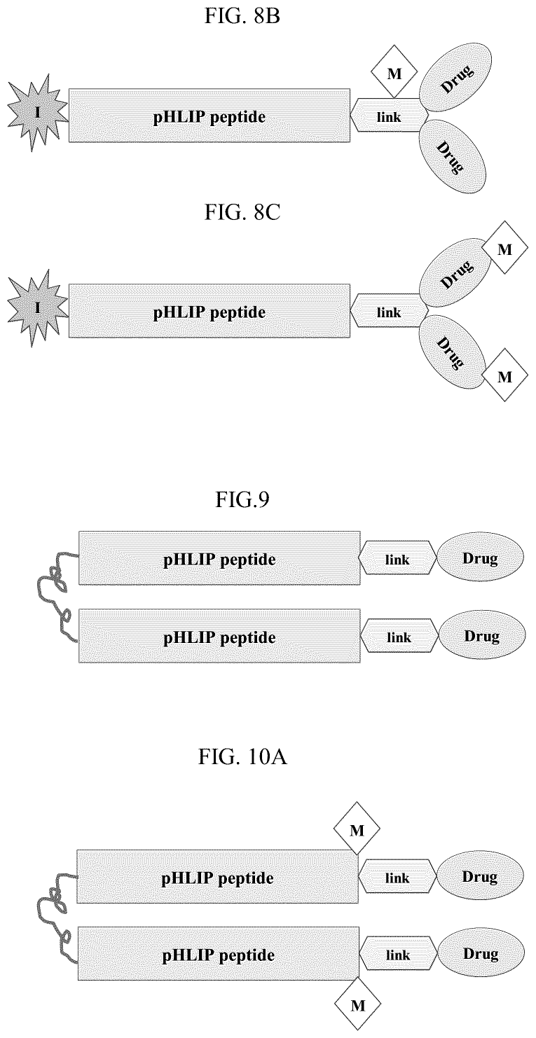

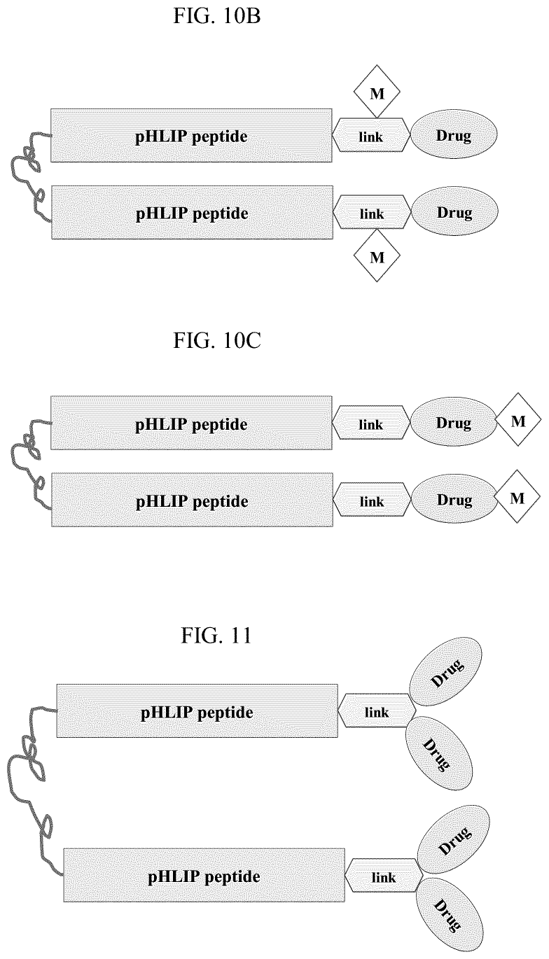

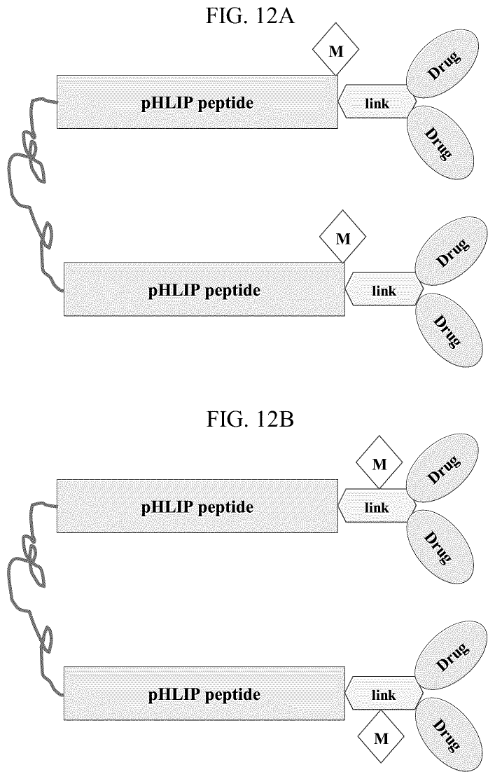

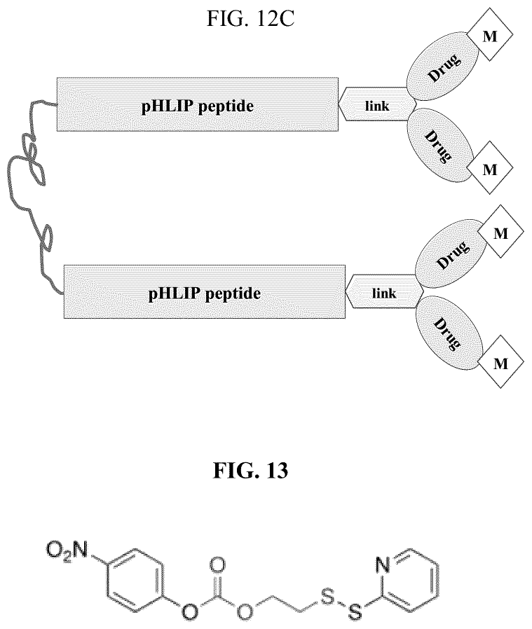

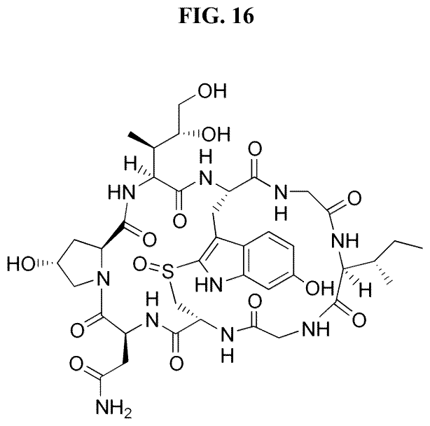

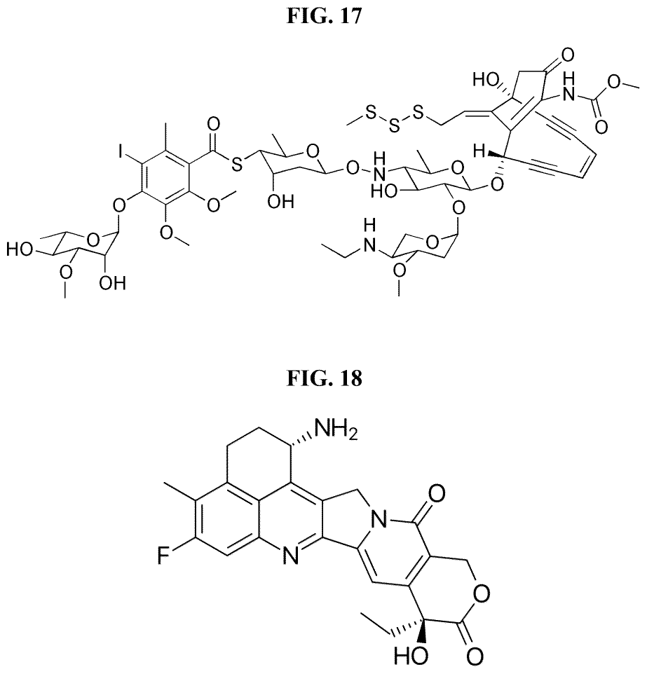

General representations of pHLIP® compounds comprising pHLIP® peptides and cytotoxic compounds are shown in FIGS. 1-16 and described below.

FIG. 1 shows a cytotoxic compound (Drug) molecule linked to a pHLIP® peptide:

The combinations shown in FIGS. 2-12 are variations of the scheme shown in FIG. 1.

One or more modulator molecules (M) are optionally attached to the pHLIP® peptide membrane-inserting end, or linker, or Drug to potentially enhance therapeutic efficacy by reducing non-specific delivery of Drug into healthy tissue (FIG. 2). A modulator molecule(s) can be a polar molecule to prevent intracellular delivery of hydrophobic and moderately hydrophobic Drug molecules across membrane of healthy cells with normal extracellular pH.

FIG. 3 shows multiple Drug molecules linked to a single pHLIP® peptide.

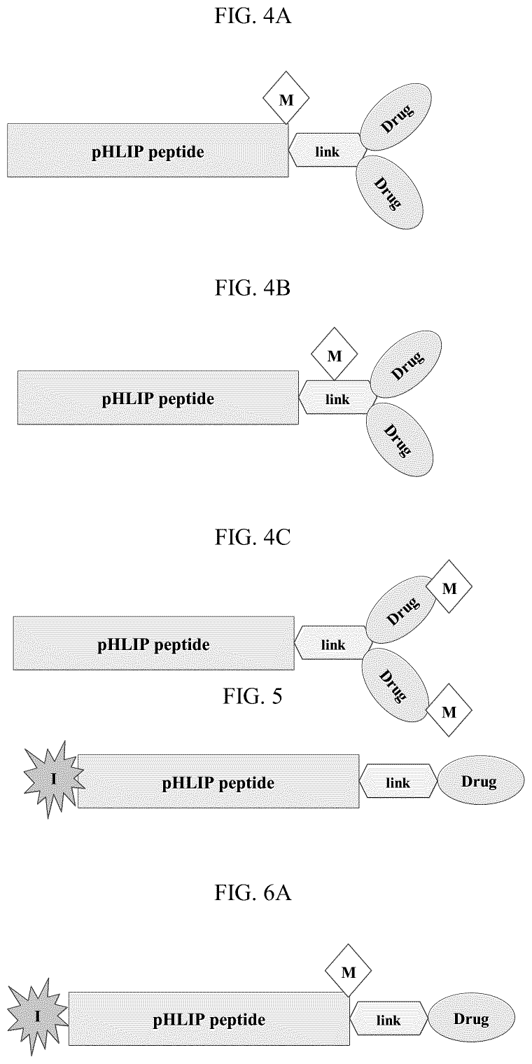

FIGS. 4A-C show multiple Drug molecules linked to a single pHLIP® peptide with one or more modulator molecules.

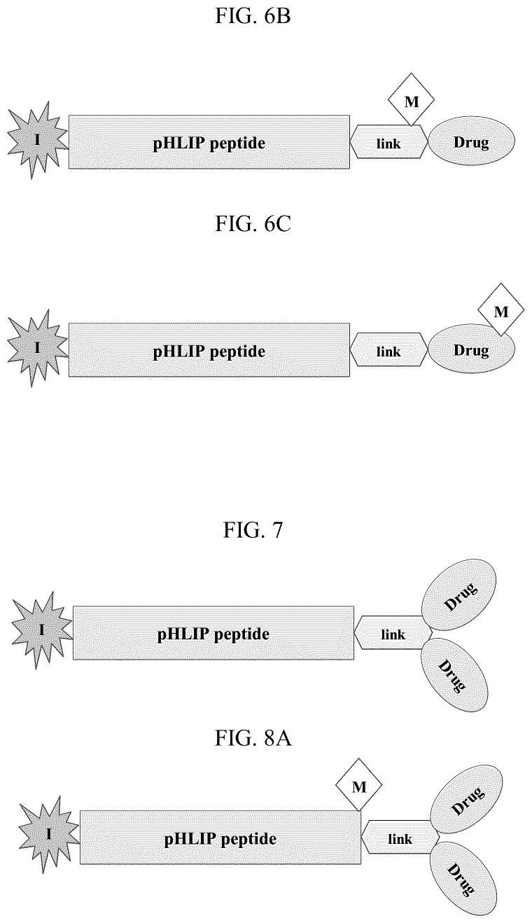

FIGS. 5, 6A-C, 7, and 8A-C depict pHLIP® compounds that can carry one or more imaging agents (I) (or other molecules) at pHLIP® peptide membrane-non-inserting end

FIG. 9 shows two or more pHLIP® peptides with a Drug cargo linked together via linker molecule

Exemplary constructs include a Var3 pHLIP® sequence ADDQNPWRAYLDLLFPTDTLLLDLLWCA (SEQ ID NO: 3) or ADDQNPWRAYLDLLFPTDTLLLDLLWKA (SEQ ID NO: 4) ADQDNPWRAYLDLLFPTDTLLLDLLWCA (SEQ ID NO: 5), or ADQDNPWRAYLDLLFPTDTLLLDLLWKA (SEQ ID NO: 6), or variations thereof, e.g., sequences provided in Tables below and in references cited herein (and incorporated by reference).

In one example, the Drug is a tubulin inhibitor molecule. A tubulin inhibitor molecule can target the tubulin system to promote tubulin polymerization thereby stabilizing microtubule structures or to inhibit tubulin polymerization thereby destabilizing microtubule structures. For example, a tubulin inhibitor molecule binds the tubulin maytansine site, resulting in the suppression of microtubule dynamics and causes cell cycle arrest in the G2/M phase. Non-limiting examples are maytansine, ansamitocin, maytansinol, D-alanyl maytansine, and maytansine derivatives and analogs (DM1, DM3 and DM4) bearing disulfide or thiol groups and their derivatives. Exemplary of maytansinoids is mertansine (or S-methyl-DM1, or DM1).

Alternatively, the Drug is an RNA polymerase inhibitor. An RNA polymerase inhibitor binds RNA polymerase and stops the production of functional RNA, including ribozymes, tRNA, control RNAs, and mRNA for protein synthesis. For example, an RNA polymerase inhibitor binds RNA polymerase II and III. Non-limiting examples include the Amanita toxins, and their derivatives and analogs. Exemplary of Amanita toxin is alpha-Amanitin.

In another example, the Drug is a DNA-damaging agent. DNA-damaging agents can cause strand scission or bind topoisomerase I-DNA complex. For example, a DNA-damaging agent binds DNA or topoisomerase I. Non-limiting examples are enediyne antibiotics and topoisomerase I inhibitors. Exemplary of enediyne antibiotics and topoisomerase I inhibitor are calicheamicin and camptothecins, their derivatives and analogs, respectively.

Drug(s) is linked to pHLIP® peptide(s) via cleavable link(s). For example, the cleavable link can be a disulfide bond, or acid-liable link. In other examples, the cleavable link is a self-immolating link.

### Maytansinoids

The potent maytansinoid and auristatin cytotoxic tubulin inhibitors have poor therapeutic windows (low therapeutic indexes) due to a lack of tumor targeting combined with high toxicity. The low therapeutic indexes have led to failures of these potent tubulin inhibitors as chemotherapeutic anticancer agents. For example, maytansine used as anti-cancer drug exhibited significant dose-limiting toxicities including neurotoxicity, gastrointestinal toxicity, weakness, nausea, vomiting, and diarrhea. Only targeted therapy can address these limitations. Currently, maytansine- and auristatin-derivatives are validated payloads in ADCs and have been extensively investigated.

Maytansinoids are anti-mitotic tubulin inhibitors derived from maytansine, which bind to the maytansine site, resulting in the suppression of microtubule dynamics and cell cycle arrest in the G2/M phase. The maytansine site is a unique site on β-tubulin that is located at the longitudinal tubulin-tubulin interface in microtubules. Inhibitors binding this site readily destabilize microtubule assembly. Three distinctly different ligands that target the maytansine site have been introduced: maytansine, PM060184 and rhizoxin. Maytansine was one of the first potent cytotoxic compounds with picomolar IC₅₀ values and higher potency than taxane- and vinblastine-site inhibitors used as anti-cancer chemotherapeutic agents. Maytansine is generated from ansamitocins, which is obtained from fermenting microorganism Actinosynnema pretiosum. Maytansinoids, such as, maytansine analogs (DM1, DM3 and DM4), were introduced by employing of a semi-synthetic strategy. Mertansine (DM1 and, in some of its forms, emtansine) is one of the maytansine analogs.

Auristatins are derived from the natural product dolastatin-10 isolated from the sea hare Dolabella auricularia. Dolastatin-10 and its analogs inhibit tubulin-dependent GTP binding and block the binding of vinca alkaloids to tubulin in a noncompetitive manner. They are widely used as ADC payloads. Compounds of pHLIP® peptides of the WT family conjugated with monomethyl auristatin F (MMAF) and monomethyl auristatin E (MMAE) exhibit significant therapeutic efficacy in mouse models without overt toxicities [U.S. Patent Application No 20170267727; Burns K E, Hensley H, Robinson M K, Thévenin D. Therapeutic Efficacy of a Family of pHLIP-MMAF Conjugates in Cancer Cells and Mouse Models. Mol Pharm. 2017 14(2), 415-422; Burns K E, Robinson M K, Thévenin D. Inhibition of cancer cell proliferation and breast tumor targeting of pHLIP-monomethyl auristatin E conjugates. Mol Pharm. 2015 12(4), 1250-1258].

### Amatoxins

Alpha-amanitin belongs to the most deadly class of all the amatoxins found in several species of the mushroom genus Amanita (Amanita phalloides and A. virosa and A. bisporigera). Alpha-amanitin is a potent inhibitor of RNA polymerase II, moderate inhibitor of RNA polymerase III and weak inhibitor of RNA polymerase IV. It blocks RNA polymerase and inhibits transcription of functional RNA, including tRNA, ribozymes, miRNA, and mRNA, and thus the subsequent synthesis of proteins, leading to cell death with 48 hours. It is a polar cyclic peptide, which cannot effectively diffuse across a plasma membrane by itself, except in liver cells, which have a special transporting system for the uptake of small cyclic molecules like phallo- and amanita toxins. The anti-proliferative effect of alpha-Amanitin conjugated with an anti-EpCAM antibody was tested on human cancer cell lines and assessed in vivo in immune-compromised mice bearing subcutaneous human pancreatic carcinoma xenograft tumors. The compounds of pHLIP® peptides conjugated with alpha-amanitin tested on cancer cell lines demonstrated concentration- and pH-dependent cytotoxicity [Wyatt L C, Moshnikova A, Crawford T, Engelman D M, Andreev O A, Reshetnyak Y K. Peptides of pHLIP family for targeted intracellular and extracellular delivery of cargo molecules to tumors. Proc Natl Acad Sci USA. 2018, 115 (12), E2811-E2818; Moshnikova A, Moshnikova V, Andreev O A, Reshetnyak Y K. Antiproliferative effect of pHLIP-amanitin. Biochemistry. 2013, 52 (7), 1171-1178]. Recent studies have indicated some tumors will be especially susceptible to alpha-amanitin treatment, since genomic deletion of the TP53 gene (that encodes the p53 protein) in some cancers is associated with suppression of neighboring genes, such as POLR2A gene, which encodes the catalytic subunit of RNA polymerase II. For example, POLR2A expression levels are tightly correlated with its gene copy numbers in human colorectal cancer. Thus, the use of alpha-Amanitin for potential treatment of colorectal cancer (or similar cancers with hemizygous TP53 and POLR2A deletions) is expected to be especially effective.

### Calicheamicins

Calicheamicin compounds belong to the chemical group of naturally occurring enediyne antibiotics that damage DNA by double-strand cleavage. Calicheamicin was originally isolated from the bacterium Micromonospora echinospora and has been recognized as the most potent antitumor agent yet identified. Calicheamicin gamma is one the most active compounds used in ADCs. Calicheamicins inside a cell undergo reductive bond cleavage by glutathione, followed by spontaneous cyclization and generation of diradicals, which subsequently form abstract hydrogen atoms from DNA, resulting in a double-strand diradical. In the presence of oxygen, DNA double strands are cleaved, followed by cell death. Several ADCs employing calicheamicin as their payload are currently being tested in clinical trials.

### Camptothecin Analogs

Camptothecin is a DNA topoisomerase I inhibitor isolated from the Chinese ornamental tree Camptothecaacuminata. Irinotecan (camptothecin-11) is a semisynthetic analog of camptothecin. Active metabolites of irinotecan known as exatecan mesylate (SN38 and DX-8951f) are used as payloads for ADCs. DX-8951f is a more water-soluble camptothecin analog, which is not an MDR1 substrate. Several ADCs employing camptothecin analogs and derivatives as their payload are currently being tested in clinical trials and obtained designation of breakthrough therapy.

### pHLIP® Peptides

An example of a wild type pHLIP® peptide (WT) is AEQNPIYWARYADWLFTTPLLLLDLALLVDADEGT (SEQ ID NO: 7) in which AEQNPIY (SEQ ID NO: 8) represents a flanking sequence, WARYADWLFTTPLLLLDLALLV (SEQ ID NO: 9) represents a membrane-inserting sequence, and DADEGT (SEQ ID NO: 10) represents a flanking sequence.

Other exemplary pHLIP® peptides are shown in the Tables below.

TABLE 1
Exemplary pHLIP ® peptides

| Name | Sequence | SEQ ID No. |
|---|---|---|
| Var3-1a | ADQDNPWRAYLDLLFPTDTLLLDLLWCA | SEQ. ID NO: 12 |
| Var3-1b | ADQDNPWRAYLDLLFPTDTLLLDLLWKA | SEQ. ID NO: 13 |
| WT-1 | GGEQNPIYWARYADWLFTTPLLLLDLALLVDADEGT | SEQ. ID NO. 14 |
| WT-2 | AEQNPIYWARYADWLFTTPLLLLDLALLVDADEGT | SEQ. ID NO. 15 |
| Var3-CK | ADDQNPWRAYLDLLFPTDTLLLDLLWCKA | SEQ. ID NO. 16 |
| Var3-WT-Cys | ADDQNPWRAYLDLLFPTDTLLLDLLWDADECG | SEQ. ID NO. 17 |
| Var3-WT-Lys | ADDQNPWRAYLDLLFPTDTLLLDLLWDADEKG | SEQ. ID NO. 18 |
| Var3-WT-KC | ADDQNPWRAYLDLLFPTDTLLLDLLWDADEKCG | SEQ. ID NO. 19 |
| Cys-Var3-WT | ACDDQNPWRAYLDLLFPTDTLLLDLLWDADEG | SEQ. ID NO. 20 |
| Lys-Var3-WT | AKDDQNPWRAYLDLLFPTDTLLLDLLWDADEG | SEQ. ID NO. 21 |
| WT-Cys1 | AAEQNPIYWARYADWLFTTPLLLLDLALLVDADEGTCG | SEQ. ID NO. 22 |
| WT-Cys2 | Ac-AEQNPIYWARYADWLFTTPLLLLDLALLVDADEGCT | SEQ. ID NO: 23 |
| WT-Cys3 | GGEQNPIYWARYADWLFTTPLLLLDLALLVDADEGTCG | SEQ. ID NO. 24 |
| Cys-WT1 | Ac-ACEQNPIYWARYADWLFTTPLLLLDLALLVDADEGTG | SEQ. ID NO. 25 |
| Var0-NT | ACEQNPIYWARYADWLFTTPLLLLDLALLVDADEGT | SEQ. ID NO. 26 |
| Lys-WT1 | AKEQNPIYWARYADWLFTTPLLLLDLALLVDADEGT | SEQ. ID NO. 27 |
| Lys-WT2 | Ac-AKEQNPIYWARYADWLFTTPLLLLDLALLVDADEGTG | SEQ. ID NO: 28 |
| WT-KC | AAEQNPIYWARYADWLFTTPLLLLDLALLVDADEGTKCG | SEQ. ID NO. 29 |
| K-WT-C | AKEQNPIYWARYADWLFTTPLLLLDLALLVDADECT | SEQ. ID NO. 30 |
| N-pHLIP | ACEQNPIYWARYANWLFTTPLLLLNLALLVDADEGTG | SEQ. ID NO. 31 |
| N-pHLIP-b | ACEQNPIYWARYANWLFTTPLLLLNLALLVDADEGT | SEQ. ID NO: 32 |
| K-pHLIP | ACEQNPIYWARYAKWLFTTPLLLLKLALLVDADEGTG | SEQ. ID NO. 33 |
| NNQ | GGEQNPIYWARYADWLFTTPLLLLDLALLVNANQGT | SEQ. ID NO. 34 |
| D25A | AAEQNPIYWARYADWLFTTPLLLLALALLVDADEGT | SEQ. ID NO. 35 |
| D25A-KC | Ac-AAEQNPIYWARYADWLFTTPLLLLELALLVDADEGTKCG | SEQ. ID NO: 36 |
| D14A | AAEQNPIYWARYAAWLFTTPLLLLDLALLVDADEGT | SEQ. ID NO. 37 |
| P20A | AAEQNPIYWARYADWLFTTALLLLDLALLVDADEGT | SEQ. ID NO. 38 |
| D25E | AAEQNPIYWARYADWLFTTPLLLLELALLVDADEGT | SEQ. ID NO. 39 |
| D14E | AAEQNPIYWARYAEWLFTTPLLLLDLALLVDADEGT | SEQ. ID NO. 40 |
| 3D | AAEQNPIIYWARYADWLFTDLPLLLLDLLALLVDADEGT | SEQ. ID NO. 41 |
| R11Q | GEQNPIYWAQYADWLFTTPLLLLDLALLVDADEGTCG | SEQ. ID NO. 42 |
| D25Up | GGEQNPIYWARYADWLFTTPLLLDLLALLVDADEGTCG | SEQ. ID NO. 43 |
| D25Down | GGEQNPIYWARYADWLFTTPLLLLLDALLVDADEGTCG | SEQ. ID NO. 44 |
| D14Up | GGEQNPIYWARYDAWLFTTPLLLLDLALLVDADEGTCG | SEQ. ID NO. 45 |
| D14Down | GGEQNPIYWARYAWDLFTTPLLLLDLALLVDADEGTCG | SEQ. ID NO. 46 |
| P20G | AAEQNPIYWARYADWLFTTGLLLLDLALLVDADEGT | SEQ. ID NO. 47 |
| H1-Cys | DDDEDNPIYWARYADWLFTTPLLLLHGALLVDADECT | SEQ. ID NO. 48 |
| H1 | DDDEDNPIYWARYADWLFTTPLLLLHGALLVDADET | SEQ. ID NO: 49 |
| H2-Cys | DDDEDNPIYWARYAHWLFTTPLLLLHGALLVDADEGCT | SEQ. ID NO. 50 |
| Cys-H2 | CDDDEDNPIYWARYAHWLFTTPLLLLHGALLVDADET | SEQ. ID NO: 51 |
| H2 | DDDEDNPIYWARYAHWLFTTPLLLLHGALLVDADEGT | SEQ. ID NO: 52 |
| H2N-Cys | DDDEDNPIYWARYAHWLFTTPLLLLHGALLVNADECT | SEQ. ID NO. 53 |
| H2N | DDDEDNPIYWARYAHWLFTTPLLLLHGALLVNADEGT | SEQ. ID NO: 54 |
| H2N2-Cys | DDDEDNPIYWARYAHWLFTTPLLLLHGALLVNANECT | SEQ. ID NO. 55 |
| H2N2 | DDDEDNPIYWARYAHWLFTTPLLLLHGALLVNANEGT | SEQ. ID NO: 56 |
| 1a-Trp | AEQNPIYWARYADFLFTTPLLLLDLALLVDADET | SEQ. ID NO. 57 |
| 1b-Trp | AEQNPIYFARYADWLFTTPLLLLDLALLVDADEGT | SEQ. ID NO. 58 |
| 1c-Trp | AEQNPIYFARYADFLFTTPLLLLDLALLWDADET | SEQ. ID NO. 59 |
| Fast-1 or Var1 | AKEDQNPYWARYADWLFTTPLLLLDLALLVDG | SEQ. ID NO. 60 |
| Var1-2D1D | ACEDQNPYWARYADWLFTTPLLLLDLALLVDG | SEQ. ID NO. 61 |
| Fastl-Cys or | AEDQNPYWARYADWLFTTPLLLLDLALLVDCG | SEQ. ID NO. 62 |
| Var1-2D1D-Cys | | |
| Fast1-E-Cys or | AEDQNPYWARYADWLFTTPLLLLELALLVECG | SEQ. ID NO. 63 |
| Var1E | | |
| Fast1-E-Lys | AKEDQNDPYWARYADWLFTTPLLLLDLALLVG | SEQ. ID NO: 64 |
| Fast2 or Var2 | AKEDQNPYWRAYADLFTPLTLLDLLALWDG | SEQ. ID NO. 65 |
| Fast2-E-Cys or | AEDQNPYWARYADWLFTTPLLLLELALLVCG | SEQ. ID NO: 66 |
| Var2E | | |
| Var2-2D1D | ACEDQNPYWRAYADLFTPLTLLDLLALWDG | SEQ. ID NO. 67 |
| Var3-3D | ACDDQNPWRAYLDLLFPTDTLLLDLLW | SEQ. ID NO. 68 |
| Var3-3D-cys | AKDDQNPWRAYLDLLFPTDTLLLDLLWC | SEQ. ID NO: 69 |
| Var4-3E | ACEEQNPWRAYLELLFPTETLLLELLW | SEQ. ID NO: 70 |
| Var5-3Da | ACDDQNPWARYLDWLFPTDTLLLDL | SEQ. ID NO: 71 |
| Var6-3Db | CDNNNPWRAYLDLLFPTDTLLLDW | SEQ. ID NO: 72 |
| Var8-3Eb | CEEQQPWAQYLELLFPTETLLLEW | SEQ. ID NO: 73 |
| Var9-3Ec | CEEQQPWRAYLELLFPTETLLLEW | SEQ. ID NO: 74 |
| Var15-2N | CDDDDDNPNYWARYANWLFTTPLLLLNGALLVEAEET | SEQ. ID NO: 75 |
| Var16-2P | CDDDDDNPNYWARYAPWLFTTPLLLLPGALLVEAEE | SEQ. ID NO: 76 |

TABLE 2
Exemplary pHLIP ® peptides

| Name | Sequence | SEQ ID No. |
|---|---|---|
| Var14-Rev | Ac-TEDADVLLALDLLLLPTTFLWDAYRAWYPNQECA-Am | SEQ. ID NO. 77 |
| Sh | AEQNPIYWARYADWLFTTPL | SEQ. ID NO. 78 |
| Sh-Cys | AEQNPIYWARYADWLFTTPCL | SEQ. ID NO. 79 |
| Cys-Sh | ACEQNPIYWARYADWLFTTPL | SEQ. ID NO. 80 |
| Sh-1Trp | AEQNPIYFARYADWLFTTPL | SEQ. ID NO. 81 |
| Sh-W2 | AEQNPIYFARYADLLFPTTLAW | SEQ. ID NO: 82 |
| Sh-W1 | AEQNPIYWARYADLLFPTTLAF | SEQ. ID NO: 83 |
| Sh-2W | AEQNPIYWARYADLLFPTTLAW | SEQ. ID NO: 84 |
| Sh-1D | KEDQNPWARYADLLFPTTLAW | SEQ. ID NO. 85 |
| Sh-1Db | KEDQNPWARYADLLFPTTLW | SEQ. ID NO: 86 |
| Var12-1D | ACEDQNPWARYADLLFPTTLAW | SEQ. ID NO. 87 |
| Var10-2D | ACEDQNPWARYADWLFPTTLLLLD | SEQ. ID NO. 88 |
| Var13-1E | ACEEQNPWARYAELLFPTTLAW | SEQ. ID NO. 89 |
| Var11-2E | ACEEQNPWARYAEWLFPTTLLLLE | SEQ. ID NO. 90 |
| Var7-3E | ACEEQNPWARYLEWLFPTETLLLEL | SEQ. ID NO. 91 |
| Var7-3Eb | ACEEQNPQAEYAEWLFPTTLLLLE | SEQ. ID NO: 92 |

“Ac” means Acetylated N-terminus
“Am” means Amidated C-terminus

TABLE 3
Coded and exemplary non-coded amino acids including L-isomers,
D-isomers, alpha-isomers, beta-isomers, glycol-,
and methyl- modifications.

| No. | Abbrev | Name |
|---|---|---|
| 1 | Ala | Alanine |
| 2 | Arg | Arginine |
| 3 | Asn | Asparagine |
| 4 | Asp | Aspartic acid |
| 5 | Cys | Cysteine |
| 6 | Gln | Glutamine |
| 7 | Glu | Glutamic acid |
| 8 | Gly | Glycine |
| 9 | His | Histidine |
| 10 | Ile | Isoleucine |
| 11 | Leu | Leucine |
| 12 | Lys | Lysine |
| 13 | Met | Methionine |
| 14 | Phe | Phenylalanine |
| 15 | Pro | Proline |
| 16 | Ser | Serine |
| 17 | Thr | Threonine |
| 18 | Trp | Tryptophan |
| 19 | Tyr | Tyrosine |
| 20 | Val | Valine |
| 21 | Sec | Selenocysteine |
| 22 | Sem | Selenomethionine |
| 23 | Pyl | Pyrrolysine |
| 24 | Aad | Alpha-aminoadipic acid |
| 25 | Acpa | Amino-caprylic acid |
| 26 | Aecys | Aminoethyl cysteine |
| 27 | Afa | Aminophenyl acetate |
| 28 | Gaba | Gamma-aminobutyric acid |
| 29 | Aiba | Aminoisobutyric acid |
| 30 | Aile | Alloisoleucine |
| 31 | AIg | Allylglycine |
| 32 | Aba | Amino-butyric acid |
| 33 | Aphe | Amino-phenylalanine |
| 34 | Brphe | Bromo-phenylalanine |
| 35 | Cha | Cyclo-hexylalanine |
| 36 | Cit | Citrulline |
| 37 | Clala | Chloroalanine |
| 38 | Cie | Cycloleucine |
| 39 | Clphe | Fenclonine (or chlorophenylalanine) |
| 40 | Cya | Cysteic acid |
| 41 | Dab | Diaminobutyric acid |
| 42 | Dap | Diaminopropionic acid |
| 43 | Dap | Diaminopimelic acid |
| 44 | Dhp | Dehydro-proline |
| 45 | Dhphe | DOPA (or 3,4-dihydroxyphenylalanine) |
| 46 | Fphe | Fluorophenylalanine |
| 47 | Gaa | Glucosaminic acid |
| 48 | Gla | Gamma-carboxyglutamic acid |
| 49 | Hag | Homoarginine |
| 50 | Hlys | Hydroxylysine |
| 51 | Hnvl | Hydroxynorvaline |
| 52 | Hog | Homoglutamine |
| 53 | Hoph | Homophenylalanine |
| 54 | Has | Homoserine |
| 55 | Hse | Homocysteine |
| 56 | Hpr | Hydroxyproline |
| 57 | Iphe | Iodo-phenylalanine |
| 58 | Ise | Isoserine |
| 59 | Mle | Methyl-leucine |
| 60 | Msmet | Methionine-methylsulfonium chloride |
| 61 | Nala | Naphthyl-alanine |
| 62 | Nle | Norleucine (or 2-aminohexanoic acid) |
| 63 | Nmala | N-methyl-alanine |
| 64 | Nva | Norvaline (or 2-aminopentanoic acid) |
| 65 | Obser | O-benzyl-serine |
| 66 | Obtyr | O-benzyl-tyro sine |
| 67 | Oetyr | O-ethyl-tyrosine |
| 68 | Omser | O-methyl-serine |
| 69 | Omthr | O-methy-threonine |
| 70 | Omtyr | O-methyl-tyrosine |
| 71 | Orn | Ornithine |
| 72 | Pen | Penicillamine |
| 73 | Pga | Pyroglutamic acid |
| 74 | Pip | Pipecolic acid |
| 75 | Sar | Sarcosine |
| 76 | Tfa | Trifluoro-alanine |
| 77 | Thphe | Hydroxy-Dopa |
| 78 | Vig | Vinylglycine |
| 79 | Aaspa | Amino-aminoethylsulfanylpropanoic acid |
| 80 | Ahdna | Amino-hydroxy-dioxanonanolic acid |
| 81 | Ahoha | Amino-hydroxy-oxahexanoic acid |
| 82 | Ahsopa | Amino-hydroxyethylsulfanylpropanoic acid |
| 83 | Tyr(Me) | Methoxyphenyl-methylpropanyl oxycarbonylamino |
| | | propanoic acid |
| 84 | MTrp | Methyl-tryptophan |
| 85 | pTyr | Phosphorylated Tyr |
| 86 | pSer | Phosphorylated Ser |
| 87 | pThr | Phosphorylated Thr |
| 88 | BLys | BiotinLys |
| 89 | Hyp | Hydroproline |
| 90 | Phg | Phenylglycine |
| 91 | Cha | Cyclohexyl-alanine |
| 92 | Chg | Cyclohexylglycine |
| 93 | Nal | Naphthylalanine |
| 94 | Pal | Pyridyl-alanine |
| 95 | Pra | Propargylglycine |
| 96 | Gly(allyl) | Pentenoic acid |
| 97 | Pen | Penicillamine |
| 98 | MetO | Methionine sulfoxide |
| 99 | Pca | Pyroglutamic acid |
| 100 | Ac-Lys | Acetylation of Lys |

TABLE 4
Non-limiting examples of protonatable residues and their substitutions
including L-isomers, D- isomers, alpha-isomers, and beta-isomers.

| Original Residue | Exemplary amino acids substitution |
|---|---|
| Asp (D) | Glu (E); Gla (Gla); Aad (Aad) |
| Glu (E) | Asp (D); Gla (Gla); Aad (Aad) |

TABLE 5
Examples of coded amino acid substitutions

| Original Residue | Substitution |
|---|---|
| Ala (A) | Gly; Ile; Leu; Met; Phe; Pro; Trp; Tyr; Val |
| Arg (R) | Lys |
| Asn (N) | Gln; His |
| Asp (D) | Glu |
| Cys (C) | Ser; Met |
| Gln (Q) | Asn; His |
| Glu (E) | Asp |
| Gly (G) | Ala; Ile; Leu; Met; Phe; Pro; Trp; Tyr; Val |
| His (H) | Asn; Gln |
| Ile (I) | Ala; Gly; Leu; Met; Phe; Pro; Trp; Tyr; Val |
| Leu (L) | Ala; Gly; Ile; Met; Phe; Pro; Trp; Tyr; Val |
| Lys (K) | Arg |
| Met (M) | Ala; Gly; Leu; Ile; Phe; Pro; Trp; Tyr; Val |
| Phe (F) | Ala; Gly; Leu; Ile; Met; Pro; Trp; Tyr; Val |
| Pro (P) | Ala; Gly; Leu; Ile; Met; Phe; Trp; Tyr; Val |
| Ser (S) | Thr |
| Thr (T) | Ser |
| Trp (W) | Ala; Gly; Leu; Ile; Met; Pro; Phe; Tyr; Val |
| Tyr (Y) | Ala; Gly; Leu; Ile; Met; Pro; Phe; Trp; Val |
| Val (V) | Ala; Gly; Leu; Ile; Met; Pro; Phe; Trp; Tyr |

```
TABLE 6
Non-limiting examples of membrane-inserting
sequences belonging to different groups of
pHLIP ® peptides. Each protonatable residue
  (shown in bold) could be replaced by its
substitution from Table 4. Each
non-polar residue could be replaced
    by its coded amino acid substitution
  from Table 5, and/or non-coded amino
acid substitutions from Table 3.
```

| Groups | Sequences | SEQ ID NO: |
|---|---|---|
| WT-BRC | WARYADWLFTTPLLLLDLALL | SEQ ID NO: 93 |
| | YARYADWLFTTPLLLLDLALL | SEQ ID NO: 94 |
| | WARYSDWLFTTPLLLYDLGLL | SEQ ID NO: 95 |
| | WARYTDWFTTPLLLYDLALLA | SEQ ID NO: 96 |
| | WARYTDWLFTTPLLLYDLGLL | SEQ ID NO: 97 |
| | WARYADWLFTTPLLLLDLSLL | SEQ ID NO: 98 |
| WT-BRC | LLALDLLLLPTTFLWDAYRAW | SEQ ID NO: 99 |
| Reverse | LLALDLLLLPTTFLWDAYRAY | SEQ ID NO: 100 |
| | LLGLDYLLLPTTFLWDSYRAW | SEQ ID NO: 101 |
| | ALLALDYLLLPTTFWDTYRAW | SEQ ID NO: 102 |
| | LLGLDYLLLPTTFLWDTYRAW | SEQ ID NO: 103 |
| | LLSLDLLLLPTTFLWDAYRAW | SEQ ID NO: 104 |
| ATRAM | GLAGLLGLEGLLGLPLGLLEGLWLGL | SEQ ID NO: 105 |
| ATRAM | LGLWLGELLGLPLGLLGELGLLGALG | SEQ ID NO: 106 |
| Reverse | | |
| Var3 | WRAYLDLLFPTDTLLLDLLW | SEQ ID NO: 107 |
| Var3 | WLLDLLLTDTPFLLDLYARW | SEQ ID NO: 108 |
| Reverse | | |
| Var7 | WARYLEWLFPTETLLLEL | SEQ ID NO: 109 |
| | WAQYLELLFPTETLLLEW | SEQ ID NO: 110 |
| Var7 | LELLLTETPFLWELYRAW | SEQ ID NO: 111 |
| Reverse | WELLLTETPFLLELYQAW | SEQ ID NO: 112 |
| Single | WLFTTPLLLLNGALLVE | SEQ ID NO: 113 |
| D/E | WLFTTPLLLLPGALLVE | SEQ ID NO: 114 |
| | WARYADLLFPTTLAW | SEQ ID NO: 115 |
| Single | EVLLAGNLLLLPTTFLW | SEQ ID NO: 116 |
| D/E | EVLLAGPLLLLPTTFLW | SEQ ID NO: 117 |
| Reverse | WALTTPFLLDAYRAW | SEQ ID NO: 118 |
| pHLIP ®- | NLEGFFATLGGEIALWSLVVLAIE | SEQ ID NO: 119 |
| Rho | EGFFATLGGEIALWSDVVLAIE | SEQ ID NO: 120 |
| | EGFFATLGGEIPLWSDVVLAIE | SEQ ID NO: 121 |
| pHLIP ®- | EIALVVLSWLAIEGGLTAFFGELN | SEQ ID NO: 122 |
| Rho | EIALVVDSWLAIEGGLTAFFGE | SEQ ID NO: 123 |
| Reverse | EIALVVDSWLPIEGGLTAFFGE | SEQ ID NO: 124 |
| pHLIP ®- | ILDLVFGLLFAVTSVDFLVQW | SEQ ID NO: 125 |

CA9

| | | |
|---|---|---|
| pHLIP ®- | WQVLFDVSTVAFLLGFVLDLI | SEQ ID NO: 126 |
| CA9 | | |
| Reverse | | |

TABLE 7
Non-limiting examples of pHLIP ® sequences. A cysteine, a lysine,
an azido-modified amino acid, or an alkynyl modified amino acid
can be incorporated at the N-terminal (first 6 residues) or
C-terminal (last 6 residues) parts of the peptides for
conjugation with a cargo, and a linker.

| SEQ ID NO | Name | Sequence |
|---|---|---|
| SEQ ID NO: 127 | WT-2D | AEQNPIYWARYADWLFTTPLLLLDLALLVDADET |
| SEQ ID NO: 128 | WT-6E | AEQNPIYWARYAEWLFTTPLLLLELALLVEAEET |
| SEQ ID NO: 129 | WT-3D | ADDQNPWRAYLDLLFPDTTDLLLLDLLWDADET |
| SEQ ID NO: 130 | WT-9E | AEEQNPWRAYLELLFPETTELLLLELLWEAEET |
| SEQ ID NO: 131 | WT-GlaD | AEQNPIYWARYAGlaWLFTTPLLLLDLALLVDADET |
| SEQ ID NO: 132 | WT-DGla | AEQNPIYWARYADWLFTTPLLLLGlaLALLVDADET |
| SEQ ID NO: 133 | WT-2Gla | AEQNPIYWARYAGlaWLFTTPLLLLGlaLALLVDADET |
| SEQ ID NO: 134 | WT-AadD | AEQNPIYWARYAAadWLFTTPLLLLDLALLVDADET |
| SEQ ID NO: 135 | WT-DAad | AEQNPIYWARYADWLFTTPLLLLAadLALLVDADET |
| SEQ ID NO: 136 | WT-2Aad | AEQNPIYWARYAAadWLFTTPLLLLAadLALLVDADET |
| SEQ ID NO: 137 | WT-GlaAad | AEQNPIYWARYAGlaWLFTTPLLLLAadLALLVDADET |
| SEQ ID NO: 138 | WT-AadGla | AEQNPIYWARYAAadWLFTTPLLLLGlaLALLVDADET |
| SEQ ID NO: 139 | WT-1 | GGEQNPIYWARYADWLFTTPLLLLDLALLVDADEGT |
| SEQ ID NO: 140 | WT-2 | GGEQNPIYWARYADWLFTTPLLLLDLALLVDADEGT |
| SEQ ID NO: 141 | WT-3 | AAEQNPIYWARYADWLFTTPLLLLDLALLVDADEGT |
| SEQ ID NO: 142 | WT-4 | AEQNPIYWARYADWLFTTPLLLLDLALLVDADEGT |
| SEQ ID NO: 143 | WT-2N | AEQNPIYWARYANWLFTTPLLLLNLALLVDADEGT |
| SEQ ID NO: 144 | WT-2K | AEQNPIYWARYAKWLFTTPLLLLKLALLVDADEGT |
| SEQ ID NO: 145 | WT-2DNANQ | GGEQNPIYWARYADWLFTTPLLLLDLALLVNANQGT |
| SEQ ID NO: 146 | WT-D25A | AAEQNPIYWARYADWLFTTPLLLLALALLVDADEGT |
| SEQ ID NO: 147 | WT-D14A | AAEQNPIYWARYAAWLFTTPLLLLDLALLVDADEGT |
| SEQ ID NO: 148 | WT-P20A | AAEQNPIYWARYADWLFTTALLLLDLALLVDADEGT |
| SEQ ID NO: 149 | WT-D25E | AAEQNPIYWARYADWLFTTPLLLLELALLVDADEGT |
| SEQ ID NO: 150 | WT-D14E | AAEQNPIYWARYAEWLFTTPLLLLDLALLVDADEGT |
| SEQ ID NO: 151 | WT-3D-2 | AAEQNPIIYWARYADWLFTDLPLLLLDLLALLVDADEGT |
| SEQ ID NO: 152 | WT-R11Q | GEQNPIYWAQYADWLFTTPLLLLDLALLVDADEG |
| SEQ ID NO: 153 | WT-D25Up | GGEQNPIYWARYADWLFTTPLLLDLLALLVDADEG |
| SEQ ID NO: 154 | WT-D25Down | GGEQNPIYWARYADWLFTTPLLLLLDALLVDADEG |
| SEQ ID NO: 155 | WT-D14Up | GGEQNPIYWARYDAWLFTTPLLLLDLALLVDADEGT |
| SEQ ID NO: 156 | WT-D14Down | GGEQNPIYWARYAWDLFTTPLLLLDLALLVDADEG |
| SEQ ID NO: 157 | WT-P20G | AAEQNPIYWARYADWLFTTGLLLLDLALLVDADEGT |
| SEQ ID NO: 158 | WT-DH | DDDEDNPIYWARYADWLFTTPLLLLHGALLVDAD |
| SEQ ID NO: 159 | WT-2H | DDDEDNPIYWARYAHWLFTTPLLLLHGALLVDADE |
| SEQ ID NO: 160 | WT-L16H | CEQNPIYWARYADWHFTTPLLLLDLALLVDADE |
| SEQ ID NO: 161 | WT-1Wa | AEQNPIYWARYADFLFTTPLLLLDLALLVDADET |
| SEQ ID NO: 162 | WT-1Wb | AEQNPIYFARYADWLFTTPLLLLDLALLVDADE |
| SEQ ID NO: 163 | WT-1Wc | AEQNPIYFARYADFLFTTPLLLLDLALLWDADET |
| SEQ ID NO: 164 | WT-W6 | ADNNPWIYARYADLTTFPLLLLDLALLVDFDD |
| SEQ ID NO: 165 | WT-W17 | ADNNPFIYARYADLTTWPLLLLDLALLVDFDD |
| SEQ ID NO: 166 | WT-W30 | ADNNPFIYARYADLTTFPLLLLDLALLVDWDD |
| SEQ ID NO: 167 | WT-W17-P7 | ADNNPFPYARYADLTTWILLLLDLALLVDFDD |
| SEQ ID NO: 168 | WT-W39-R11 | ADNNPFIYAYRADLTTFPLLLLDLALLVDWDD |
| SEQ ID NO: 169 | WT-W30-R15 | ADNNPFIYATYADLRTFPLLLLDLALLVDWDD |
| SEQ ID NO: 170 | WT-Rev | Ac-TEDADVLLALDLLLLPTTFLWDAYRAWYPNQEA-Am |
| SEQ ID NO: 171 | Var1-3D | AEDQNPYWARYADWLFTTPLLLLDLALLVD |
| SEQ ID NO: 172 | Var1-1D2E | AEDQNPYWARYADWLFTTPLLLLELALLVE |
| SEQ ID NO: 173 | Var2-3D | AEDQNPYWRAYADLFTPLTLLDLLALWD |
| SEQ ID NO: 174 | Var3-3D | ADDQNPWRAYLDLLFPTDTLLLDLLW |
| SEQ ID NO: 175 | Var3-WT | ADDQNPWRAYLDLLFPTDTLLLDLLWDADE |
| SEQ ID NO: 176 | Var3-Gla2D | ADDQNPWRAYLGlaLLFPTDTLLLDLLW |
| SEQ ID NO: 177 | Var3-DGlaD | ADDQNPWRAYLDLLFPTGlaTLLLDLLW |
| SEQ ID NO: 178 | Var3-2DGla | ADDQNPWRAYLDLLFPTDTLLLGlaLLW |
| SEQ ID NO: 179 | Var3-2GlaD | ADDQNPWRAYLGlaLLFPTGlaTLLLDLLW |
| SEQ ID NO: 180 | Var3-GlaDGla | ADDQNPWRAYLGlaLLFPTDTLLLGlaLLW |
| SEQ ID NO: 181 | Var3-D2Gla | ADDQNPWRAYLDLLFPTGlaTLLLGlaLLW |
| SEQ ID NO: 182 | Var3-3Gla | ADDQNPWRAYLGlaLLFPTGlaTLLLGlaLLW |
| SEQ ID NO: 183 | Var3-Aad2D | ADDQNPWRAYLAadLLFPTDTLLLDLLW |
| SEQ ID NO: 184 | Var3-DAadD | ADDQNPWRAYLDLLFPTAadTLLLDLLW |
| SEQ ID NO: 185 | Var3-2DAad | ADDQNPWRAYLDLLFPTDTLLLAadLLW |
| SEQ ID NO: 186 | Var3-2AadD | ADDQNPWRAYLAadLLFPTAadTLLLDLLW |
| SEQ ID NO: 187 | Var3-AadDAad | ADDQNPWRAYLAadLLFPTDTLLLAadLLW |
| SEQ ID NO: 188 | Var3-D2Aad | ADDQNPWRAYLDLLFPTAadTLLLAadLLW |
| SEQ ID NO: 189 | Var3-3Aad | ADDQNPWRAYLAadLLFPTAadTLLLAadLLW |
| SEQ ID NO: 190 | Var3-G1aAadD | ADDQNPWRAYLGlaLLFPTAadTLLLDLLW |
| SEQ ID NO: 191 | Var3-GlaDAad | ADDQNPWRAYLGlaLLFPTDTLLLAadLLW |
| SEQ ID NO: 192 | Var3-2GlaAad | ADDQNPWRAYLGlaLLFPTGlaTLLLAadLLW |
| SEQ ID NO: 193 | Var3-AadGlaD | ADDQNPWRAYLAadLLFPTGlaTLLLDLLW |
| SEQ ID NO: 194 | Var3-AadDGla | ADDQNPWRAYLAadLLFPTDTLLLGlaLLW |
| SEQ ID NO: 195 | Var3-GlaAadGla | ADDQNPWRAYLGlaLLFPTAadTLLLGlaLLW |
| SEQ ID NO: 196 | Var3-GLL | GEEQNPWLGAYLDLLFPLELLGLLELGLW |
| SEQ ID NO: 197 | Var3-M | ADDDDDDPWQAYLDLLFPTDTLLLDLLW |
| SEQ ID NO: 198 | Var4-3E | AEEQNPWRAYLELLFPTETLLLELLW |
| SEQ ID NO: 199 | Var5-3Da | ADDQNPWARYLDWLFPTDTLLLDL |
| SEQ ID NO: 200 | Var6-3Db | DNNNPWRAYLDLLFPTDTLLLDW |
| SEQ ID NO: 201 | Var7-3E | AEEQNPWARYLEWLFPTETLLLEL |
| SEQ ID NO: 202 | Var7-M | DDDDDDPWQAYLDLFPTDTLALDLW |
| SEQ ID NO: 203 | Var8-3E | EEQQPWAQYLELLFPTETLLLEW |
| SEQ ID NO: 204 | Var9-3E | EEQQPWRAYLELLFPTETLLLEW |
| SEQ ID NO: 205 | Var10-2D | AEDQNPWARYADWLFPTTLLLLD |
| SEQ ID NO: 206 | Var11-2E | AEEQNPWARYAEWLFPTTLLLLE |
| SEQ ID NO: 207 | Var12-1D | AEDQNPWARYADLLFPTTLAW |
| SEQ ID NO: 208 | Var13-1E | AEEQNPWARYAELLFPTTLAW |
| SEQ ID NO: 209 | Var15-2N | DDDDDNPNYWARYANWLFTTPLLLLNGALLVEAEET |
| SEQ ID NO: 210 | Var16-2P | DDDDDNPNYWARYAPWLFTTPLLLLPGALLVEAEET |
| SEQ ID NO: 211 | Var17 | AEQNPIYFARYADFLFTTPLLLLDLALLWDADET |
| SEQ ID NO: 212 | Var18 | AEQNPIYWARYADFLFTTPLLLLDLALLVDADET |
| SEQ ID NO: 213 | Var19a | AEQNPIYWARYADWLFTTPL |
| SEQ ID NO: 214 | Var20 | AEQNPIYFARYADLLFPTTLAW |
| SEQ ID NO: 215 | Var21 | AEQNPIYWARYADLLFPTTLAF |
| SEQ ID NO: 216 | Var22 | AEQNPIYWARYADLLFPTTLAW |
| SEQ ID NO: 217 | Var23 | AEQNPIYFARYADWLFTTPL |
| SEQ ID NO: 218 | Var24 | EDQNPWARYADLLFPTTLAW |
| SEQ ID NO: 219 | ATRAM | GLAGLAGLLGLEGLLGLPLGLLEGLWLGLELEGN |
| SEQ ID NO: 220 | pHLIP-CA9 | EQNPIYILDLVFGLLFAVTSVDFLVQWDDAGD |
| SEQ ID NO: 221 | pHLIP-Rho | NLEGFFATLGGEIALWSLVVLAIE |
| SEQ ID NO: 222 | pHLIP-RhoM1 | NNEGFFATLGGEIALWSDVVLAIE |
| SEQ ID NO: 223 | pHLIP-RhoM2 | DNNEGFFATLGGEIPLWSDVVLAIE |

### Methods for Treating Cancer, Inflammation, Atherosclerosis, or Targeting Senescent Cells in a Subject

Included herein is a method of preventing or treating cancer, inflammation, atherosclerosis, or targeting senescent cells in a subject (e.g., killing cells in acidic diseased tissue) in a subject in need thereof. In further embodiments, the method comprises administering to the subject an effective amount of a composition comprising a pHLIP® peptide and a cytotoxic compound (e.g., a cytotoxic tubulin inhibitor, a cytotoxic RNA polymerase inhibitor, a cytotoxic DNA damaging compound, or a toposisomerase I inhibitor).

For example, methods for preventing or treating cancer, inflammation, atherosclerosis, or targeting senescent cells in a subject (e.g., killing cells in acidic diseased tissue) include administering a composition comprising an effective amount of a composition comprising a pHLIP® peptide and a cytotoxic compound.

In other embodiments, the methods for treating cancer, inflammation, atherosclerosis, or targeting senescent cells in a subject comprises administering to a subject a composition comprising a pHLIP® peptide and a cytotoxic compound produced according to the methods described herein, optionally in combination with other treatment methods. In particular, the combination treatment can include administering readily known treatments. For example, combination therapy may include hormonal, radiation and/or immuno-therapy treatment in combination with administration of pHLIP-cytotoxic molecule.

Combination therapy may also include administration of corticosteroids or pHLIP linked to corticosteroid together with administration of pHLIP-cytotoxic molecule.

The described composition can be administered as a pharmaceutically or physiologically acceptable preparation or composition containing a physiologically acceptable carrier, excipient, or diluent, and administered to the tissues of the recipient organism of interest, including humans and non-human animals.

The composition comprising a pHLIP® peptide and a cytotoxic compound can be prepared by re-suspending in a suitable liquid or solution such as sterile physiological saline or other physiologically acceptable injectable aqueous liquids. The amounts of the components to be used in such compositions can be routinely determined by those having skill in the art.

In embodiments, a therapeutically effective amount of the composition (e.g., a composition comprising a pHLIP® peptide and a cytotoxic compound) in humans can be any therapeutically effective amount. In one embodiment, the composition (e.g., a composition comprising a pHLIP® peptide and a cytotoxic compound) is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In an embodiment, the composition (e.g., a composition comprising a pHLIP® peptide and a cytotoxic compound) is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks).

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

pHLIP® peptides are described here and in U.S. Pat. No. 9,814,781 and 9,289,508 (hereby incorporated by reference) as well as U.S. Patent Publication 20180117183, 20180064648, 20180221500, 20180117183, 20180064648, 20160256560, 20150191508, 20150051153, and 20120142042, 20120039990, and 20080233107, each of which is hereby incorporated by reference.

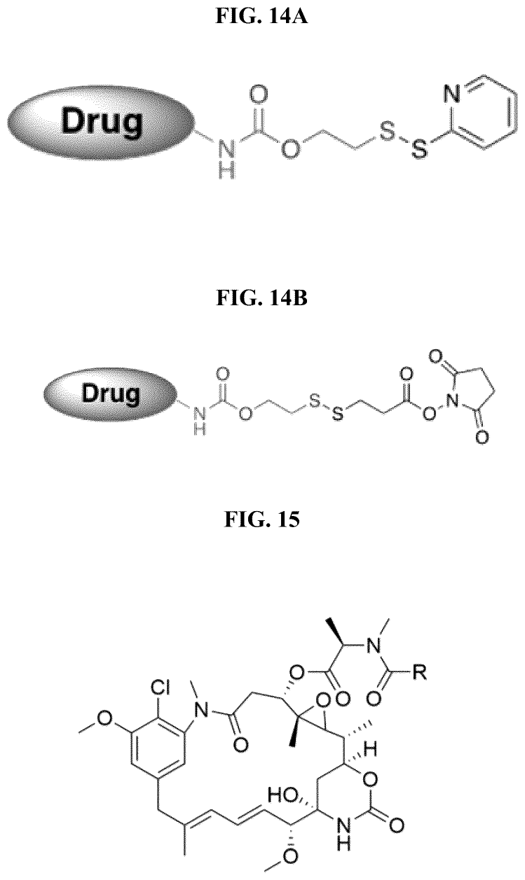

Examples of potent cytotoxic compounds include: Maytansinoids, their derivatives and analogs, which are exemplary of tubulin inhibitors; alpha-amanitin, its derivatives and analogs, which are exemplary of RNA polymerase inhibitors; calicheamicins, their derivatives and analogs, which are exemplary of DNA-damaging agents; and camptothecins, their derivatives and analogs, which are exemplary of DNA-damaging topoisomerase I inhibitors. The use of these potent cytotoxic compounds has been hampered by delivery issues. FIGS. 13-14A-B show exemplary pHLIP®-linker-Drug constructs. As described above, it has not been possible to achieve an acceptable therapeutic window in the use of maytansinoids, alpha-amanitin, calicheamicins, camptothecins, and their derivates and analogs as anti-cancer agents alone, so targeted delivery is required. Constructs described here, e.g., a pHLIP®-linker-Drug construct, mediate targeting of maytansinoids, alpha-amanitin, calicheamicins, camptothecins, and their derivates and analogs to tumor cells (due to their surface low pH) and reduce delivery of these potent cytotoxic compounds to normal cells (with normal extracellular pH), thus avoiding or minimizing side effects, increasing the therapeutic window and enhancing the therapeutic index. The problem of targeting a potent cytotoxic compound, which is in this case maytansinoids, or alpha-amanitin, calicheamicins, or camptothecins, or the derivates and analogs of these compounds, is solved by the pHLIP®/drug compositions of the invention.

A linker could be relatively small, e.g., only a few atoms, or rather large (4-5 kDa). FIG. 13A show an exemplary heterobifunctional linker that reacts on one end with a free thiol to spontaneously form a disulfide bond, with thiopyridine as a leaving group, and on the other end reacts with activated with amine or hydroxyl groups in the presence of DIPEA, and in some cases DMAP or other activator base, to form a carbamate or carbonate, respectively.

FIG. 14A is a linker-drug conjugate that reacts with a free thiol at pHLIP® peptide bearing a cysteine residue to spontaneously form a disulfide bond, with thiopyridine as a leaving group. FIG. 14B is a linker-drug conjugate that reacts with a free amino group at pHLIP® bearing a lysine residue, if pHLIP® is protected at its amino terminus, such as with N-acetylation.

In some examples, the following cross-linkers can be used: SPDP (succinimidyl 3-(2-pyridyldithio)propionate); LC-SPDP (succinimidyl 6-(3(2-pyridyldithio)propionamido)hexanoate); sulfo-LC-SPDP (sulfosuccinimidyl 6-(3′-(2-pyridyldithio)propionamido)hexanoate); PEG4-SPDP (PEGylated, long-chain SPDP crosslinker); PEG12-SPDP (PEGylated, long-chain SPDP crosslinker); SMCC (succinimidyl 4-(N-maleimidomethyl)cyclohexane-1-carboxylate); sulfo-SMCC (sulfosuccinimidyl 4-(N-maleimidomethyl)cyclohexane-1-carboxylate); SMPT (4-succinimidyloxycarbonyl-alpha-methyl-α(2-pyridyldithio)toluene); DTME (dithiobismaleimidoethane). The invention may encompass the following embodiments.

A compound having the formula:

Peptide—Mod—Linker—Drug (1),

wherein:
Peptide is a pHLIP®,
Mod is a modulator, and is optional. Mod comprises a chemical entity to modulate the overall polarity of a Linker-Drug for optimized targeting by pHLIP®. To achieve optimized targeting the overall polarity of Mod-Linker-Drug can be guided by LogP, where P is the measured octanol-water partition coefficient, is preferably in the range −1<LogP<1. Optionally, the Mod can be attached to the inserting end of pHLIP®, the linker, or the drug.

In some cases, a potent cytotoxic compound or drug is polar or moderately hydrophobic. The average value of the measured LogP for drugs is about 2-3. Exemplary drugs are polar, moderately hydrophobic or hydrophobic as defined by the following characteristics. Polar: LogP<−0.4; Moderately hydrophobic: 2.5<LogP<−0.4; and Hydrophobic: LogP>2.5. The polarity and/or hydrophobicity of a drug or compound to be delivered is measured using methods known in the art, e.g., by determining LogP, in which P is the octanol-water partition coefficient. A substance is dissolved into an octanol-water mixture, mixed, and allowed to come to equilibration. The amount of substance in each (or one) phases is then measured. The measurements itself could be in a number of ways known in the art, e.g., by measuring absorbance, or determining the amount using NMR, HPLC, or other known methods. If the cargo is polar (LogP<−0.4), the hydrophobic modulator will increase the LogP of (the cargo-modulator) (LogP>−0.4). If cargo is hydrophobic LogP>2.5, the polar modulator will decrease LogP of [cargo-modulator] (LogP<2.5).

Linker comprises a covalent bond or a chemical linker such that (1) is selected from the following:

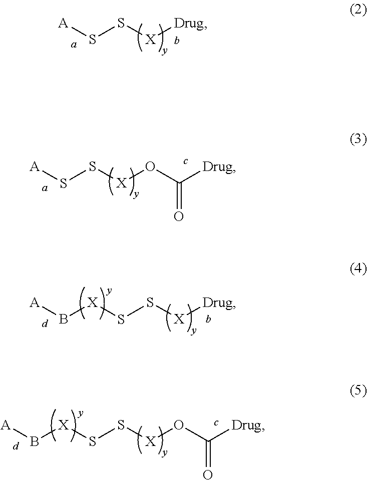

each occurrence of y may be present or absent and is independently an integer ranging from 1 to 4;

each occurrence of X is independently selected from the group consisting of CH₂, CH(alkyl), and C(alkyl)₂;

each occurrence of B may be present or absent and is independently selected from the group consisting of alkyl, aryl, and PEG;

bond a is formed between the sulfur and the thiol substituent of a cysteine residue in A;

bond b is formed between the carbon and a substituent on the Drug, wherein the substituent is selected from the group consisting of hydroxyl, carbonyl, amine, amide, sulfate, sulfonamide, phosphate, and phosphoramide;

bond c is formed between the carbonyl and a substituent on Drug, wherein the substituent is selected from the group consisting of primary amine, secondary amine, and hydroxyl;

bond d is formed between B and an amino acid residue in A, wherein the amino acid is selected from the group consisting of serine, threonine, tyrosine, tryptophan, histidine, lysine, and cysteine and comprises an amide, ester, carbamate, carbonate, or maleimide bond.

Drug comprises or consists of a potent cytotoxic drug or compound with anticancer activity. The potent cytotoxic compound (or a high-potency active pharmaceutical ingredient, HPAPI) is defined as an agent with an occupational exposure limit (OEL) at or below 10 μg/m³ of air as an 8-h time-weighted average and a pharmacologically active ingredient or intermediate with high selectivity and/or with the potential to cause cancer, mutations, developmental effects, or reproductive toxicity at low doses.

Exemplary drugs are described below. The Drug is a tubulin inhibitor, which binds one of the following tubulin binding sites: maytansine, vinca, taxane, colchicine, pironetin, and laulimalide/peloruside. The composition of formula (1) may include a Drug that is a tubulin inhibitor. Alternatively, the Drug is selected from the group consisting of tubulin inhibitors and their derivatives including microtubule destabilizers: maytansine and its derivatives, dolastatin-10 and its derivatives, cryptopycin-1, cryptopycin-52, tubulysins D, hemiasterlin and its derivative (HTI-286), colchicine and CA4, and microtubule stabilizers: paclitaxel, discodermolide, taccalonolides A and B and their derivatives (taccalonolide AF and taccalonolide AJ), and taccalonolide AI-epoxide, laulimalide, and epothilones A and B:

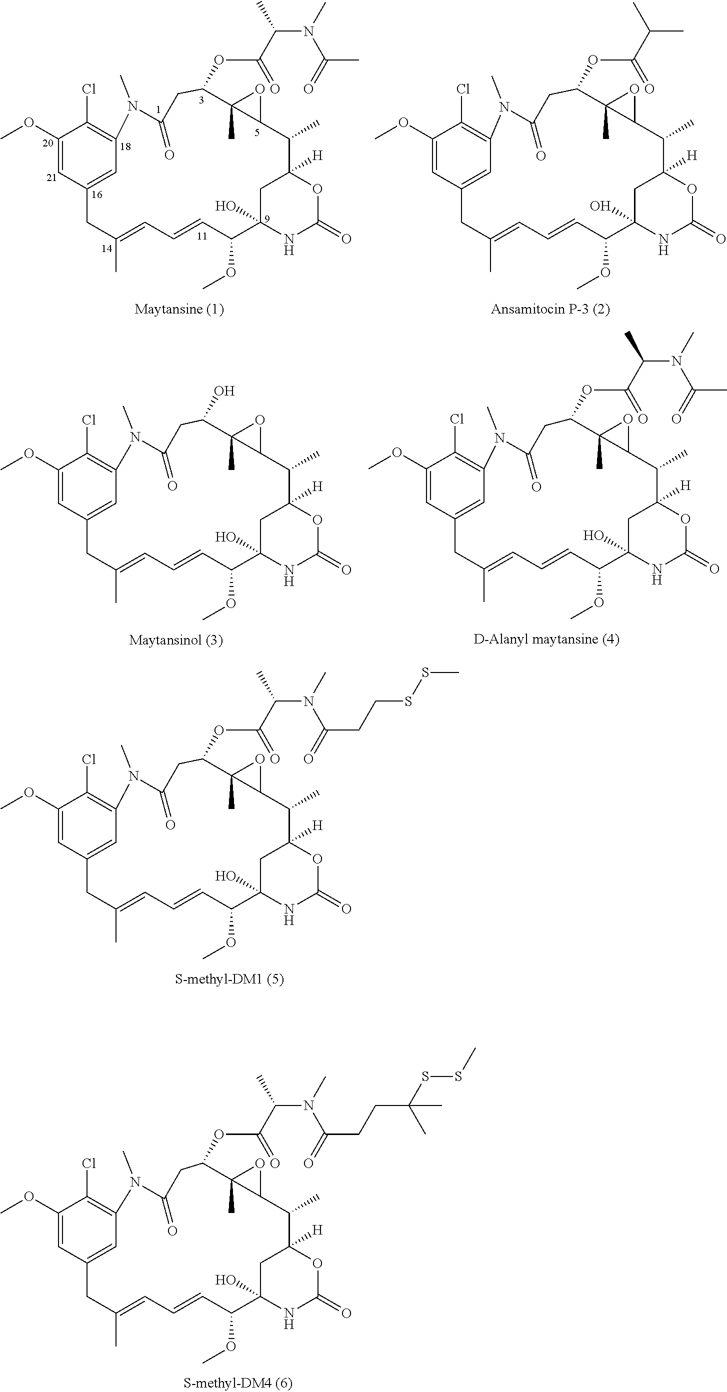

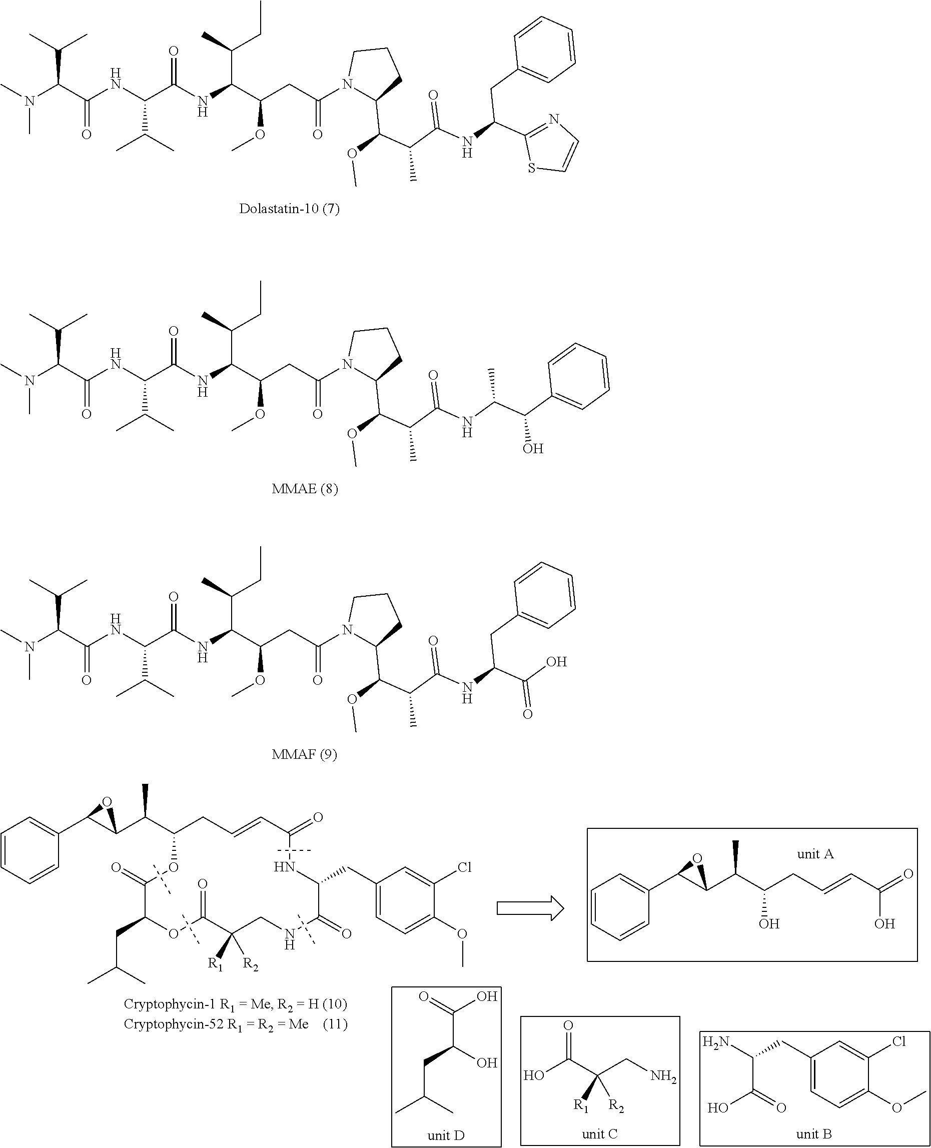

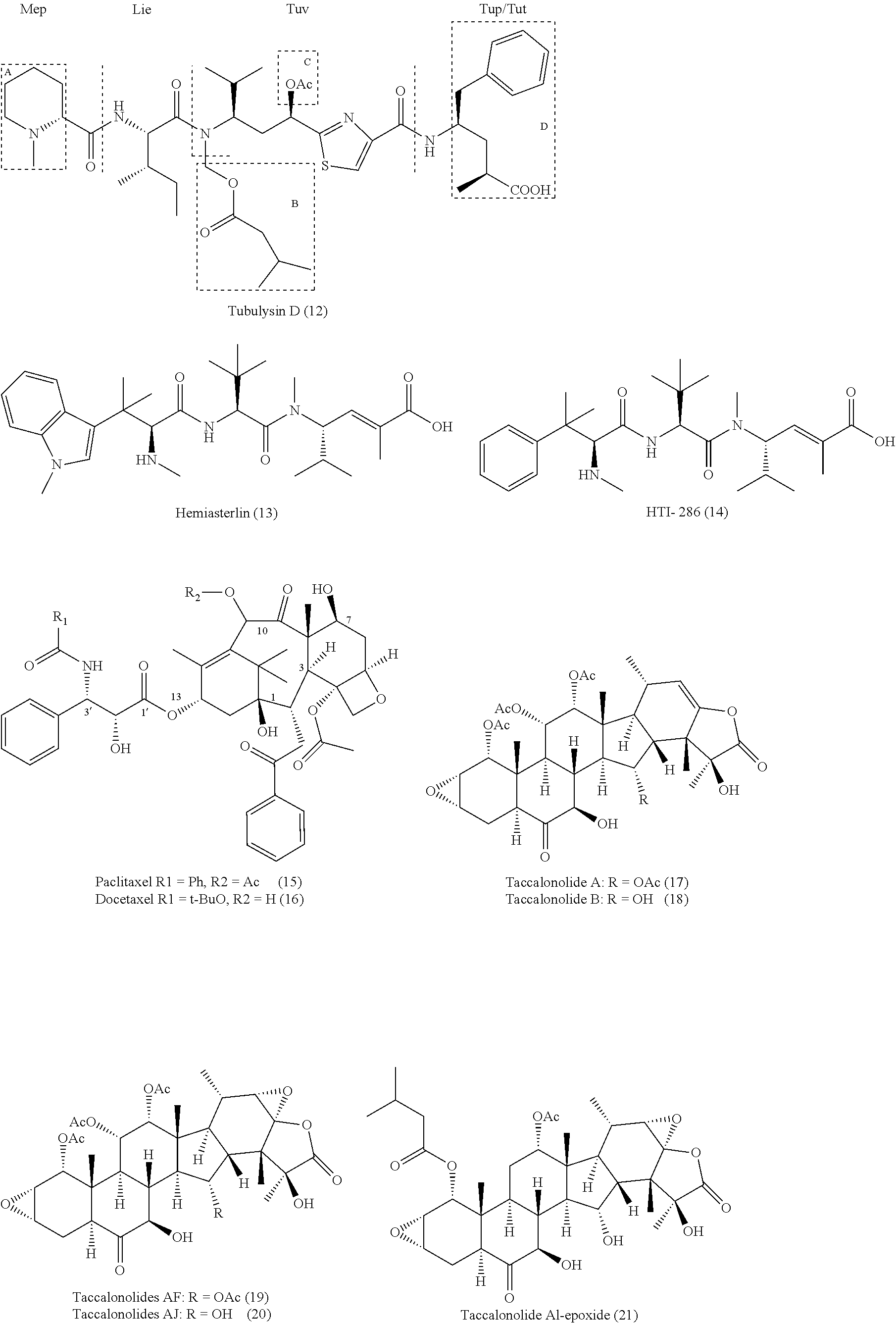

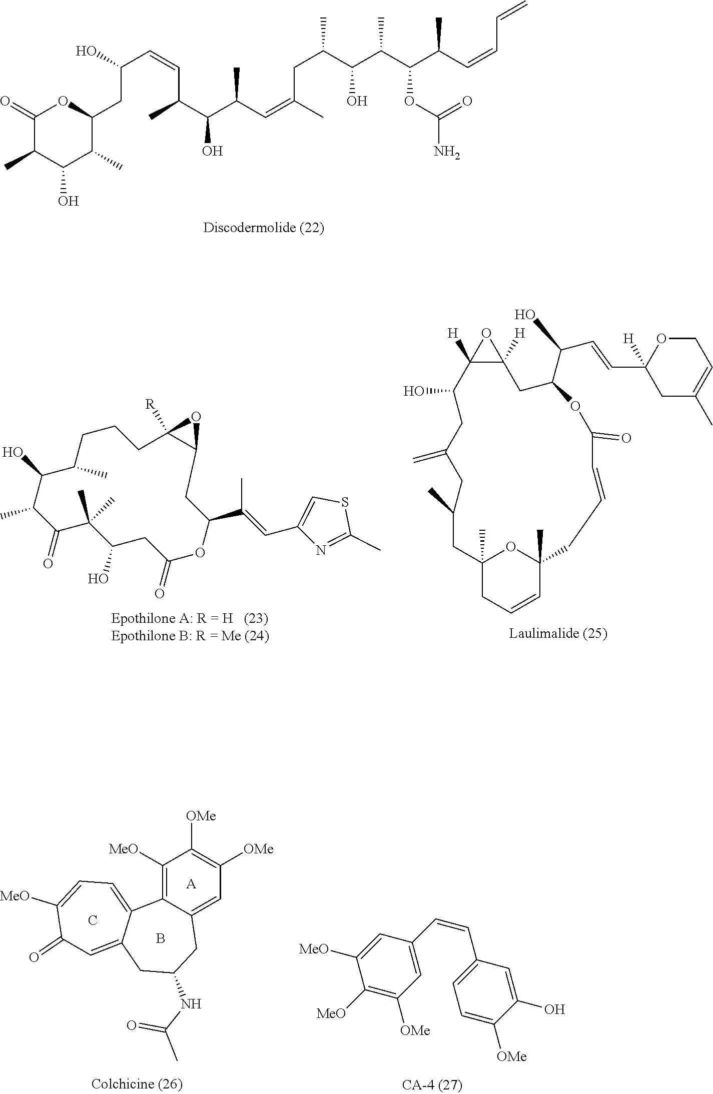

A compound of formula (1), wherein Drug is a maytansinoid, a tubulin inhibitor, which binds maytansine tubulin binding site.

A compound of formula (1), wherein Drug is selected from the group consisting of maytansinoids:

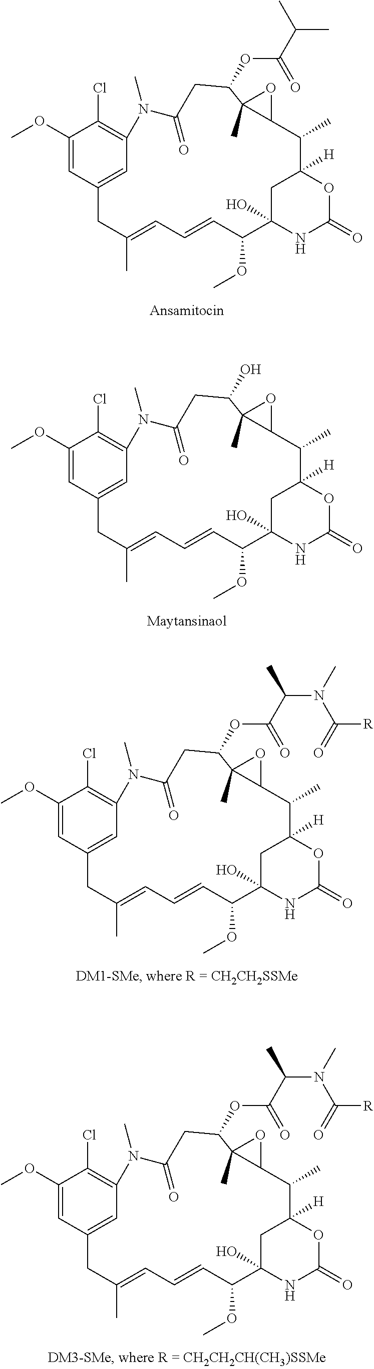

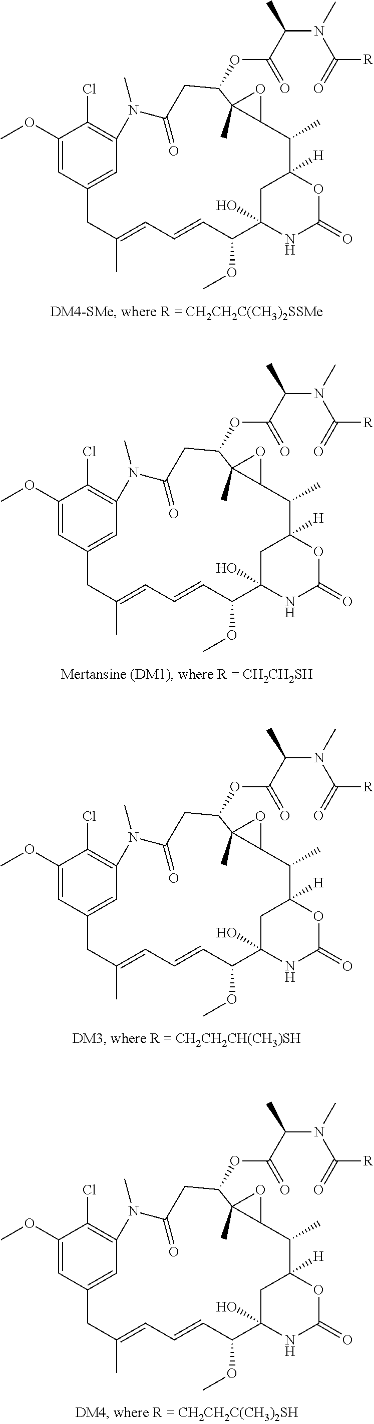

and their analogs and derivatives.

Drug is an amatoxin, which binds RNA polymerase II.

A compound of formula (1), wherein Drug is an amatoxin. The sequence of amatoxins is Ile-Trp-Gly-Ile-Gly-Cys-Asn-Pro (SEQ ID NO: 11) with cross-linking between Trp and Cys via the sulfoxide (S═O) moiety.

A compound of formula (1), wherein Drug is selected from the group consisting of α-amanitin, β-amanitin, γ-amanitin, ε-amanitin, amanullin, amanullinic acid, amaninamide, amanin, and proamanullin:

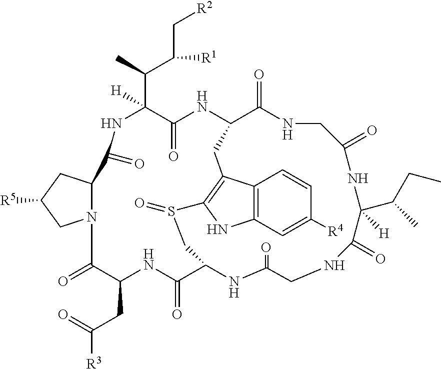

where α-amanitin (R¹═OH; R²═OH; R³═NH₂; R⁴═OH; R⁵═OH), β-amanitin (R¹═OH; R²═OH; R³═OH; R⁴═OH; R⁵═OH), γ-amanitin (R¹═OH; R²═H; R³═NH₂; R⁴═OH; R⁵═OH), ε-amanitin (R¹═OH; R²═H; R³═OH; R⁴═OH; R⁵═OH), amanullin (R¹═H; R²═H; R³═NH₂; R⁴═OH; R⁵═OH), amanullinic acid (R¹═H; R²═H; R³═OH; R⁴═OH; R⁵═OH), amaninamide (R¹═OH; R²═OH; R³═NH₂; R⁴═H; R⁵═OH), amanin (R¹═OH; R²═OH; R³═OH; R⁴═H; R⁵═OH), and proamanullin (R¹═H; R²═H; R³═NH₂; R⁴═OH; R⁵═H).

A compound of formula (1), wherein Drug is an alpha-amanitin:

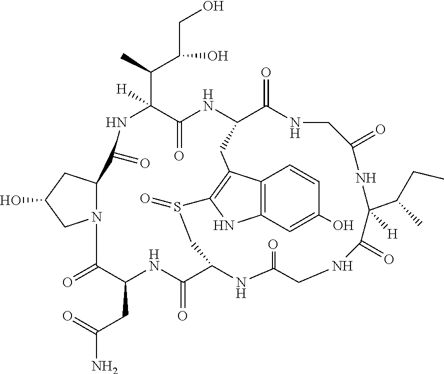 [SMILES: CC[C@H](C)[C@@H]1NC(=O)CNC(=O)[C@@H]2Cc3c([nH]c4cc(O)ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

and its analogs and derivatives.

Drug is a DNA-damaging agent, which binds minor groove of DNA and cause strand scission.

A compound of formula (1), wherein Drug is an enediyne antibiotic.

A compound of formula (1), wherein Drug is calicheamicin:

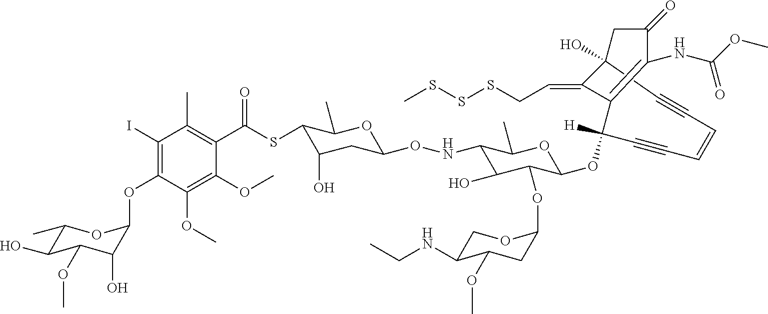 [SMILES: CCNC1COC(OC2C(O[C@H]3C#C/C=C\C#C[C@]4(O)CC(=O)C(NC(=O)OC)=C3/C4=C\CSSSC)OC(C)C(NOC3CC(O)C(SC(=O)c4c(C)c(I)c(OC5OC(C)C(O)C(OC)C5O)c(OC)c4OC)C(C)O3)C2O)CC1OC]

and its analogs and derivatives.

Drug is a DNA-damaging agent, which binds topoisomerase I.

A compound of formula (1), wherein Drug is selected from the group consisting of camptothecin compounds:

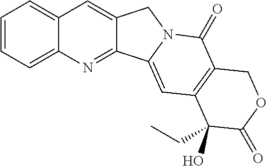 [SMILES: CC[C@@]1(O)C(=O)OCc2c1cc1n(c2=O)Cc2cc3ccccc3nc2-1]

A compound of formula (1), wherein Drug is an exatecan (DX-8951f):

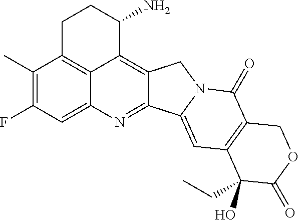 [SMILES: CC[C@@]1(O)C(=O)OCc2c1cc1n(c2=O)Cc2c-1nc1cc(F)c(C)c3c1c2[C@@H](N)CC3]

and its analogs and derivatives.

### Example 2: pHILIP®-Mediated Tumor Targeting and Cytoplasmic Delivery of Cytotoxic Tubulin Inhibitor, Mertansine, Group of Inhibitors of Tubulin's Maytansine Site

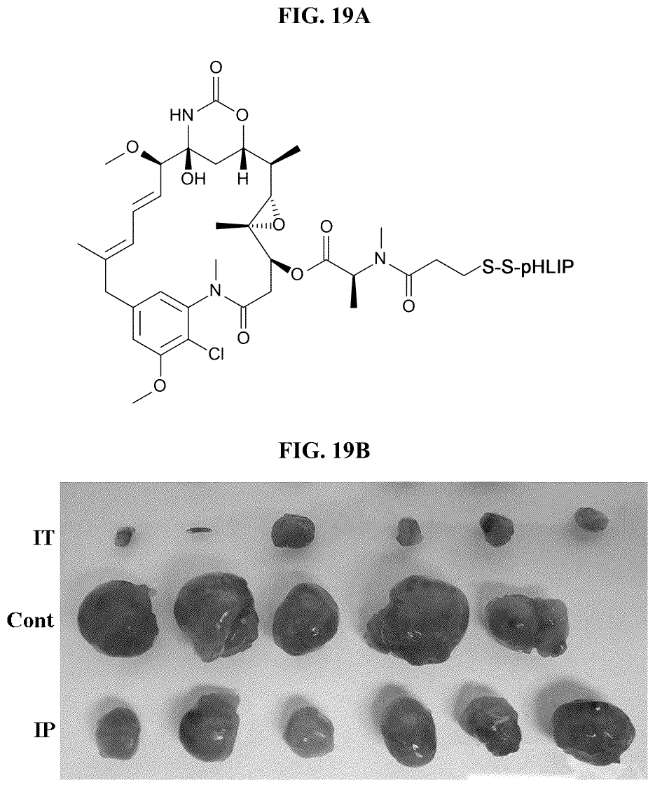

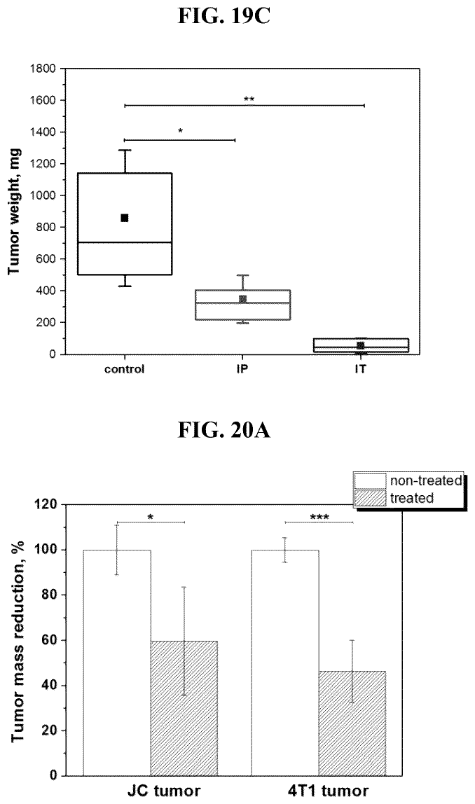

Mertansine (DM1; CAS No: 139504-50-0) was conjugated to pHILIP® peptide membrane-inserting end via cleavable S—S link (FIG. 19A). The pHILIP® peptide used in the study: ADDQNPWRAYLDLLFPTDTLLLDLLWCA (SEQ ID NO: 3) was prepared by solid-phase synthesis. First, pHLIP-Cys was conjugated with Aldrithiol™-2(2,2′-Dipyridyldisulfide; CAS No: 2127-03-9) to obtain PHILIP-S—S-Pyridine. pHLIP-S—S-Pyridine is purified by the reverse phase HPLC (the gradient: water and acetonitrile with 0.05% TFA). Then, mertansine was conjugated with pHLIP-S—S-Pyridine to exchange S—S bond and obtain pHLIP-S—S-mertansine followed by purification by RP-HPLC (the gradient: water and acetonitrile with 0.05% TFA) and lyophilization. The construct purity and identity was established by analytical RP-HPLC and surface-enhanced laser desorption/ionization time of flight (SELDI-TOF) mass spectroscopy, respectively. The construct concentration was calculated by absorbance at 280 nm for pHLIP® peptide with normalized from the absorbance of mertansine (see FIG. 19A).

pHLIP-S—S-mertansine dissolved in PBS and was given as multiple intraperitoneal (IP) or intratumoral (IT) injections (once in 3-4 days) into mice bearing MDA-MB-231 human triple negative breast or HeLa human cervical tumors in flanks of female athymic nude mice. The total combined dose of the multiple IP injections of pHLIP-S—S-mertansine was ˜10 mg/kg, and the total combined dose of the multiple IT injections of pHLIP-S—S-mertansine was ˜2.5 mg/kg. When the tumor reached a size of about 1 cm³ (about 1 g) in the control (non-treated) group, the animals were sacrificed; tumors were collected (FIG. 19B) and weighted (FIG. 19C).

About 60% of tumor weight reduction was observed after IP administration of pHLIP-S—S-mertansine, and more than 90% of tumor weight reduction was observed after IT administration of pHLIP-S—S-mertansine. In some cases the tumor disappeared after IT administration of the construct.

In a separate experiment, IT injections were performed, tumors disappeared and mice were left for several weeks. In 60% of these cases, no tumor re-growth was observed, and no signs of toxicity were observed in the course of IP or IT treatment.

### Example 3: pHLIP®-Mediated Tumor Targeting and Cytoplasmic Delivery of Cytotoxic RNA Polymerase Inhibitor, Alpha-Amanitin, Group of Amatoxins

Alpha-amanitin was conjugated to a pHLIP® peptide membrane-inserting end via cleavable S—S link. The pHLIP® peptide used in the study: ADDQNPWRAYLDLLFPTDTLLLDLLWCA (SEQ ID NO: 3) was prepared by solid-phase synthesis. To prepare pHLIP-S—S-amanitin, a-amanitin was conjugated to succinimidyl 3-(2-piridyldithio)propionate (SPDP) crosslinker, followed by purification using RP-HPLC (the gradient: water and acetonitrile with 0.05% TFA) and conjugation of the SPDP-amanitin to the C-terminal cysteine (bold and underlined in the sequence above) residues of pHLIP® peptide. Progression of the reaction and purification of pHLIP-S—S-amanitin was conducted using RP-HPLC (the gradient: water and acetonitrile with 0.05% TFA), followed lyophilization. The construct purity and identity was established by analytical RP-HPLC and surface-enhanced laser desorption/ionization time of flight (SELDI-TOF) mass spectroscopy, respectively. The construct concentration was calculated by absorbance at 310 nm, where, for α-amanitin, ε₃₁₀=13,000 M⁻¹ cm⁻¹.

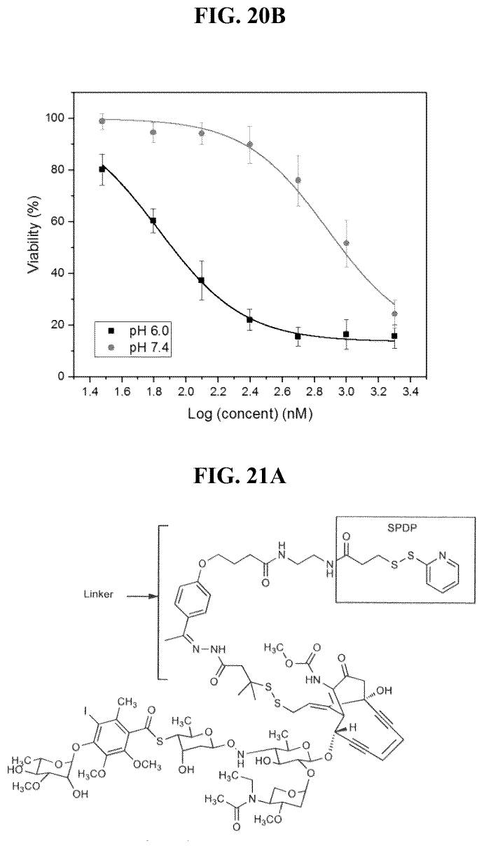

pHLIP-S—S-amanitin dissolved in PBS and was given as multiple IP injections (once in 3-4 days) into female Balb/C mice bearing JC or 4T1 murine breast tumors in flank. The total combined dose of the multiple injections of pHLIP-S—S-amanitin was ˜0.3 mg/kg. Each injection was accompanied by IP administration of 6.3 mg/kg of solution (200 μL) of glucose 30 min prior of pHLIP-S—S-amanitin administration. When the tumor reached a size of about 1 cm³ (about 1 g) in the control group, the animals were sacrificed, tumors were collected and weighted. About 40-50% reduction of tumor weight is observed (FIG. 20A).

The cytotoxic and highly polar agents, like amanitin, may be especially effective in topical applications. For example, the superficial bladder cancer may be treated with pHLIP-S—S-amantin, when the construct is administered via intravesical instillation. pHLIP-S—S-amanitin was tested on a panel of urinary bladder cancer cell lines at pH 7.4 and pH 6.0 to establish ratio, which demonstrated the difference in construct performance at different pHs, and, which could be interpreted as therapeutic index.

Cancer cells were loaded in the wells of 96-well plates (5,000 cells/well) and incubated overnight. The standard growth medium was replaced with medium without FBS, at pH 6.0 or 7.4, containing increasing amounts of pHLIP-S—S-amanitin (from 0 to 2.0 μM). The pH 6.0 medium was prepared by mixing 13.3 g of dry DMEM in 1 L of deionized water. After two-hour incubation with the pHLIP-S—S-amanitin, the construct was removed and replaced with standard growth medium. Treatment with amanitin alone for two hours at concentrations up to 2 μM did not induce cell death. Cell viability was assessed after 48 hours using the colorimetric CellTiter 96 AQᵤₑₒᵤₛ One Solution Cell Proliferation Assay by absorption measurement at 490 nm (FIG. 20B presents an example of the data obtained on SCaBER squamous cell carcinoma).

There was no difference in the viability of cells incubated with media, without construct, at pH 7.4 and pH 6.0; therefore, the role of pH was excluded from the consideration. Therapeutic index (TI) was calculated according to the equation:

$$T I = \frac{{EC}_{50}^{pH 7.4}}{{EC}_{50}^{pH 6.}}$$

Table 8 below contains EC₅₀ values obtained for the treatment at pH 7.4 and pH 6.0 and calculated therapeutic index, which clearly indicated a pH-dependent cell action of pHLIP-S—S-amanitin. The ratio (TI) varied from 3.5 to 8 for different cell lines. pHLIP-S—S-amanitin distinguishes, binds to, and inserts into the cell membrane of cancer cells with low cell surface pH (pH5.5-6.5) from healthy cells with normal cell surface pH (pH7.4).

TABLE 8
The EC₅₀ values in nM calculated for treatment of pHLIP-S-S-amanitin
with urinary bladder cancer cell lines at pH7.4 and pH6.0

| | EC₅₀_{_{pH6.0}} | EC₅₀_{_{pH7.4}} | TI |
|---|---|---|---|
| HT-1197 cells: urinary bladder carcinoma | 193.8 | 804.8 | 4.2 |
| 5637 cells: grade II carcinoma | 89.9 | 378.2 | 4.2 |
| HT-1376 cells: grade III carcinoma | 766.7 | 3002.3 | 3.9 |
| SCaBER cells: squamous cell carcinoma | 127.8 | 1017.0 | 8.0 |
| J82 cells: transitional cell carcinoma | 83.7 | 549.1 | 6.6 |
| UM-UC-3 cells: transitional cell carcinoma | 446.2 | 2143.0 | 4.8 |
| SW780 cells: transitional cell carcinoma | 144.9 | 887.8 | 6.1 |
| T-24 cells: transitional cell carcinoma | 163.0 | 575.4 | 3.5 |
| TCCSUR cells: IV transitional cell carcinoma | 72.1 | 391.3 | 5.4 |
| RT4 cells: transitional cell papilloma | 107.3 | 473.3 | 4.4 |

### Example 4: pHLIP®-Mediated Tumor Targeting and Cytoplasmic Delivery of Cytotoxic DNA-Damaging Compound, Calicheamicin, Group of Enediyne Antibiotic

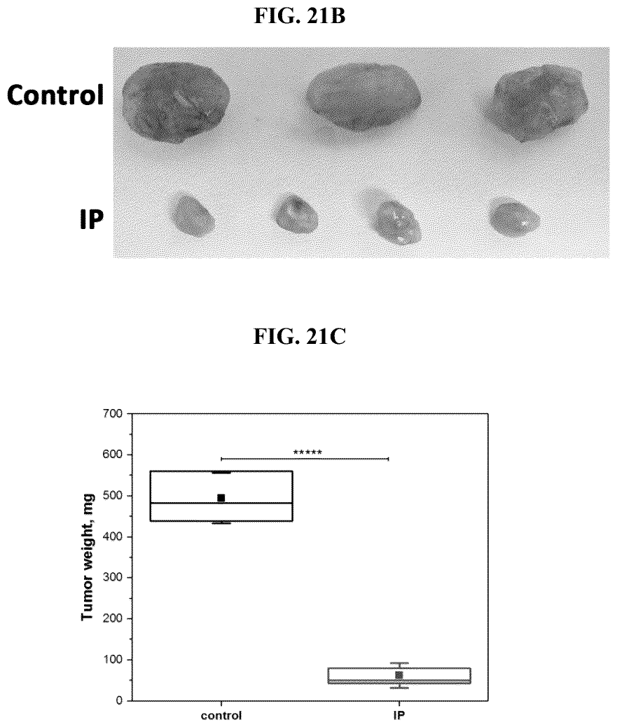

Calicheamicin modified with SPDP was synthesized and purified by Cfm, GmbH (FIG. 21A). Calicheamicin-SPDP was used to conjugate with Cys residue of PHILIP® peptide's membrane-inserting end to obtain pHLIP-S—S-calicheamicin. The pHLIP® peptide used in the study: ADDQNPWRAYLDLLFPTDTLLLDLLWCA (SEQ ID NO: 3) was prepared by solid-phase synthesis. Purification of the pHLIP-S—S-calicheamicin was conducted using RP-HPLC (the gradient: 10 mM TEAA buffer and acetonitrile), followed by lyophilization. The construct purity and identity was established by analytical RP-HPLC and surface-enhanced laser desorption/ionization time of flight (SELDI-TOF) mass spectroscopy, respectively. Construct concentration was calculated by absorbance at 280 nm for pHLIP® peptide with correction on absorbance of calicheamicin.

pHLIP-S—S-calicheamicin dissolved in PBS was given as multiple intraperitoneal (IP) or intratumoral (IT) injections (once in 3-4 days) into mice bearing HeLa human cervical tumors in flank of female athymic nude mice.

The total combined dose of the multiple IP injections of pHLIP-S—S-calicheamicin was ˜1.5 mg/kg. When tumors reached about 0.5 g in the control (non-treated) group, the animals were sacrificed; tumors were collected (FIG. 21B) and weighted (FIG. 21C).

About 88% of tumor weight reduction was observed after IP administration of pHLIP-S—S-calicheamicin

### Example 5: pHLIP®-Mediated Tumor Targeting and Cytoplasmic Delivery of Cytotoxic DNA-Damaging Compound, Topoisomerase I Inhibitor Exatecan, Group of Camptothecin Compounds

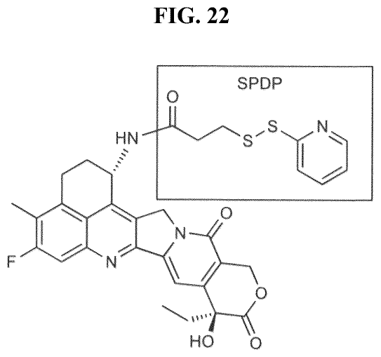

Exatecan modified with SPDP is synthesized and purified by Cfm GmbH (FIG. 22). Exatecan-SPDP is conjugated with a single Cys residue of pHLIP® peptide's membrane-inserting end to obtain pHLIP-S—S-exatecan. The pHLIP® peptide used in the study: ADDQNPWRAYLDLLFPTDTLLLDLLWCA (SEQ ID NO: 3) is prepared by solid-phase synthesis. Purification of pHLIP-S—S-exatecan is conducted using RP-HPLC followed by lyophilization. The construct purity and identity are established by analytical RP-HPLC and surface-enhanced laser desorption/ionization time of flight (SELDI-TOF) mass spectroscopy, respectively. Construct concentration was calculated by absorbance at 280 nm for pHLIP® peptide with correction on absorbance of exatecan.

pHLIP-S—S-exatecan dissolved in PBS is given as multiple intraperitoneal (IP) injections (once in 3-4 days) into mice bearing HeLa human cervical tumors in flank of female athymic nude mice. When tumors reached about 1 cm³ (about 1 g) in the control (non-treated) group, the animals are sacrificed; tumors were collected and weighted.

About 50-60% of tumor weight reduction is observed after IP administration of pHLIP-S—S-exatecan, and more than 70% of tumor weight reduction is observed after IT administration of pHLIP-S—S-exatecan.

### Example 6

In aspects, provided herein is a composition comprising a potent cytotoxic compound and a pHLIP® peptide. For example, the cytotoxic compound is a cytotoxic tubulin inhibitor compound, which inhibits tubulin polymerization and destabilization of microtubule structures. In other embodiments, the cytotoxic tubulin inhibitor compound binds to a maytansine site.

In embodiments, the cytotoxic tubulin inhibitor compound includes a maytansine, ansamitocin, maytansinol, D-alanyl maytansine, or maytansine analogs bearing disulfide or thiol groups and their derivatives. For example, the cytotoxic tubulin inhibitor compound includes mertansine or a derivative thereof.

In embodiments, the cytotoxic compound is a cytotoxic RNA polymerase inhibitor. For example, the cytotoxic RNA polymerase inhibitor compound is an amatoxins. In other examples, the cytotoxic RNA polymerase inhibitor compound is an alpha-amanitin or a derivative thereof.

In embodiments, the cytotoxic compound is a cytotoxic DNA-damaging compound. For example, the cytotoxic DNA damaging compound is an enediyne antibiotic. In other examples, the cytotoxic DNA damaging compound is calicheamicin or a derivative thereof. In embodiments, the cytotoxic DNA damaging compound is a topoisomerase I inhibitor. For example, the cytotoxic topoisomerase I inhibitor compound is a camptothecin compound. In embodiments, the cytotoxic topoisomerase I inhibitor compound is exatecan or a derivative thereof.

In embodiments, the cytotoxic compound comprises limited targeting of tumors.

In embodiments, the composition described herein further includes a linker between said cytotoxic compound and said pHLIP® peptide. For example, the linker includes a disulfide bond or an acid-liable bond. In embodiments, the linker is cleavable. In other embodiments, the linker is not cleavable. In embodiments, the linker is self-immolating.

In embodiments, the composition described herein further includes a modulator of polarity.

In embodiments, also provided herein is a composition including a pHLIP® peptide, wherein the pHLIP® peptide has the sequence ADDQNPWRAYLDLLFPTDTLLLDLLWXA (SEQ ID NO: 1) or ADQDNPWRAYLDLLFPTDTLLLDLLWXA (SEQ ID NO: 2), wherein upper case “X” indicates any amino acid residue and can include a lysine (Lys), a cysteine (Cys), or an Azido-containing amino acid.

In embodiments, the composition has the following structure: Peptide—Link—B

wherein “Peptide” is a first pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2), “B” is a second pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2), wherein upper case “X” indicates any amino acid residue and can include lysine (Lys), Cysteine (Cys), or Azido-containing amino acid; “Link” is a polyethylene glycol linker, and each “—” is a covalent bond.

In aspects, provided herein is a method of killing cells in acidic diseased tissue, comprising administering to a subject a composition comprising a cytotoxic compound and a pHLIP® peptide.

In other embodiments, the method includes that the subject has a solid tumor. For example, in embodiments, the subject has a bladder tumor.

In embodiments, the composition is injected directly into a tumor mass. In embodiments, the composition is instilled into a bladder.

In embodiments, the composition is topically applied. In other embodiments, the composition is systemically administered. In other embodiments, the tubulin inhibitor compound is delivered into the cytosols of cancer cells. In examples, the cytotoxic compound is delivered into the cytosols of senescent cells.

In embodiments, the cytotoxic compound is targeted to acidic tissue to induce a biological effect predominantly within targeted tissue.

In embodiments, the cytotoxic compound is delivered intracellularly to induce a biological effect. In other embodiments, the cytotoxic compound has limited tumor targeting ability in the absence of said pHLIP®.

In embodiments, the composition targets said cytotoxic compound preferentially to a diseased tissue compared to a healthy tissue, thereby minimizing damage to said healthy tissue.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the amino acid sequences, or nucleic acid sequences that flank it in its naturally-occurring state. Purified also defines a degree of sterility that is safe for administration to a human subject, e.g., lacking infectious or toxic agents. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A the “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

In various embodiments, an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

1. A composition comprising a potent cytotoxic compound and a pHLIP® peptide.

2. The composition of claim 1, wherein the cytotoxic compound is a cytotoxic tubulin inhibitor compound, which inhibits tubulin polymerization and destabilization of microtubule structures.

3. The composition of claim 2, wherein the cytotoxic tubulin inhibitor compound binds to a maytansine site.

4. (canceled)

5. (canceled)

6. The composition of claim 1, wherein the cytotoxic compound is a cytotoxic RNA polymerase inhibitor.

7. The composition of claim 6, wherein the cytotoxic RNA polymerase inhibitor compound comprises an amatoxin.

8. (canceled)

9. The composition of claim 1, wherein the cytotoxic compound is a cytotoxic DNA-damaging compound.

10. The composition of claim 9, wherein the cytotoxic DNA damaging compound comprises an enediyne antibiotic.

11. The composition of claim 9, wherein the cytotoxic DNA damaging compound comprises calicheamicin.

12. The composition of claim 9, wherein the cytotoxic DNA damaging compound comprises a topoisomerase I inhibitor.

13. The composition of claim 12, wherein the cytotoxic topoisomerase I inhibitor compound comprises a camptothecin compound.

14. The composition of claim 13, wherein the cytotoxic topoisomerase I inhibitor compound comprises exatecan.

15. The composition of claim 1, wherein the cytotoxic compound comprises limited targeting of tumors.

16. The composition of claim 1, further comprising a linker between the cytotoxic compound and the pHLIP® peptide.

17. The composition of claim 16, wherein the linker comprises a disulfide bond or an acid-labile bond.

18. The composition of claim 16, wherein the linker is cleavable.

19. The composition of claim 16, wherein the linker is not cleavable.

20. The composition of claim 16, wherein the linker is self-immolating.

21. The composition of claim 1, further comprising a modulator of polarity.

22. The composition of claim 1, wherein the pHLIP® peptide comprises the sequence ADDQNPWRAYLDLLFPTDTLLLDLLWXA (SEQ ID NO: 1) or ADQDNPWRAYLDLLFPTDTLLLDLLWXA (SEQ ID NO: 2), wherein upper case “X” indicates any amino acid residue and can include a lysine (Lys), a cysteine (Cys), or an Azido-containing amino acid.

23. The composition of claim 1, comprising the following structure:
Peptide—Link—B
wherein “Peptide” is a first pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2), “B” is a second pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2),
wherein upper case “X” indicates any amino acid residue and can include lysine (Lys), Cysteine (Cys), or Azido-containing amino acid; “Link” is a polyethylene glycol linker, and each “—” is a covalent bond.

24-36. (canceled)


SEQUENCE LISTING (223 sequences)

SEQ ID NO: 1 - protein, 28 aa, synthetic construct
  REGION 1..28: note = Description of Artificial Sequence: Synthetic peptide
  MOD_RES 27: note = Any amino acid
  REGION 1..28: note = See specification as filed for detailed description of substitutions and preferred embodiments
ADDQNPWRAYLDLLFPTDTLLLDLLWXA

SEQ ID NO: 2 - protein, 28 aa, synthetic construct
  REGION 1..28: note = Description of Artificial Sequence: Synthetic peptide
  MOD_RES 27: note = Any amino acid
  REGION 1..28: note = See specification as filed for detailed description of substitutions and preferred embodiments
ADQDNPWRAYLDLLFPTDTLLLDLLWXA

SEQ ID NO: 3 - protein, 28 aa, synthetic construct
  REGION 1..28: note = Description of Artificial Sequence: Synthetic peptide
ADDQNPWRAYLDLLFPTDTLLLDLLWCA

SEQ ID NO: 4 - protein, 28 aa, synthetic construct
  REGION 1..28: note = Description of Artificial Sequence: Synthetic peptide
ADDQNPWRAYLDLLFPTDTLLLDLLWKA

SEQ ID NO: 5 - protein, 28 aa, synthetic construct
  REGION 1..28: note = Description of Artificial Sequence: Synthetic peptide
ADQDNPWRAYLDLLFPTDTLLLDLLWCA

SEQ ID NO: 6 - protein, 28 aa, synthetic construct
  REGION 1..28: note = Description of Artificial Sequence: Synthetic peptide
ADQDNPWRAYLDLLFPTDTLLLDLLWKA

SEQ ID NO: 7 - protein, 35 aa, synthetic construct
  REGION 1..35: note = Description of Artificial Sequence: Synthetic polypeptide
AEQNPIYWARYADWLFTTPLLLLDLALLVDADEGT

SEQ ID NO: 8 - protein, 7 aa, synthetic construct
  REGION 1..7: note = Description of Artificial Sequence: Synthetic peptide
AEQNPIY

SEQ ID NO: 9 - protein, 22 aa, synthetic construct
  REGION 1..22: note = Description of Artificial Sequence: Synthetic peptide
WARYADWLFTTPLLLLDLALLV

SEQ ID NO: 10 - protein, 6 aa, synthetic construct
  REGION 1..6: note = Description of Artificial Sequence: Synthetic peptide
DADEGT

SEQ ID NO: 11 - protein, 8 aa, unidentified
  REGION 1..8: note = Description of Unknown: Amatoxin peptide
IWGIGCNP

SEQ ID NO: 12 - protein, 28 aa, synthetic construct
  REGION 1..28: note = Description of Artificial Sequence: Synthetic peptide
ADQDNPWRAYLDLLFPTDTLLLDLLWCA

SEQ ID NO: 13 - protein, 28 aa, synthetic construct
  REGION 1..28: note = Description of Artificial Sequence: Synthetic peptide
ADQDNPWRAYLDLLFPTDTLLLDLLWKA

SEQ ID NO: 14 - protein, 36 aa, synthetic construct
  REGION 1..36: note = Description of Artificial Sequence: Synthetic polypeptide
GGEQNPIYWARYADWLFTTPLLLLDLALLVDADEGT

SEQ ID NO: 15 - protein, 35 aa, synthetic construct; identical to SEQ ID NO: 7
  REGION 1..35: note = Description of Artificial Sequence: Synthetic polypeptide

SEQ ID NO: 16 - protein, 29 aa, synthetic construct
  REGION 1..29: note = Description of Artificial Sequence: Synthetic peptide
ADDQNPWRAYLDLLFPTDTLLLDLLWCKA

SEQ ID NO: 17 - protein, 32 aa, synthetic construct
  REGION 1..32: note = Description of Artificial Sequence: Synthetic polypeptide
ADDQNPWRAYLDLLFPTDTLLLDLLWDADECG

SEQ ID NO: 18 - protein, 32 aa, synthetic construct
  REGION 1..32: note = Description of Artificial Sequence: Synthetic polypeptide
ADDQNPWRAYLDLLFPTDTLLLDLLWDADEKG

SEQ ID NO: 19 - protein, 33 aa, synthetic construct
  REGION 1..33: note = Description of Artificial Sequence: Synthetic polypeptide
ADDQNPWRAYLDLLFPTDTLLLDLLWDADEKCG

SEQ ID NO: 20 - protein, 32 aa, synthetic construct
  REGION 1..32: note = Description of Artificial Sequence: Synthetic polypeptide
ACDDQNPWRAYLDLLFPTDTLLLDLLWDADEG

SEQ ID NO: 21 - protein, 32 aa, synthetic construct
  REGION 1..32: note = Description of Artificial Sequence: Synthetic polypeptide
AKDDQNPWRAYLDLLFPTDTLLLDLLWDADEG

SEQ ID NO: 22 - protein, 38 aa, synthetic construct
  REGION 1..38: note = Description of Artificial Sequence: Synthetic polypeptide
AAEQNPIYWARYADWLFTTPLLLLDLALLVDADEGTCG

SEQ ID NO: 23 - protein, 36 aa, synthetic construct
  REGION 1..36: note = Description of Artificial Sequence: Synthetic polypeptide
AEQNPIYWARYADWLFTTPLLLLDLALLVDADEGCT

SEQ ID NO: 24 - protein, 38 aa, synthetic construct
  REGION 1..38: note = Description of Artificial Sequence: Synthetic polypeptide
GGEQNPIYWARYADWLFTTPLLLLDLALLVDADEGTCG

SEQ ID NO: 25 - protein, 37 aa, synthetic construct
  REGION 1..37: note = Description of Artificial Sequence: Synthetic polypeptide
ACEQNPIYWARYADWLFTTPLLLLDLALLVDADEGTG

SEQ ID NO: 26 - protein, 36 aa, synthetic construct
  REGION 1..36: note = Description of Artificial Sequence: Synthetic polypeptide
ACEQNPIYWARYADWLFTTPLLLLDLALLVDADEGT

SEQ ID NO: 27 - protein, 36 aa, synthetic construct
  REGION 1..36: note = Description of Artificial Sequence: Synthetic polypeptide
AKEQNPIYWARYADWLFTTPLLLLDLALLVDADEGT

SEQ ID NO: 28 - protein, 37 aa, synthetic construct
  REGION 1..37: note = Description of Artificial Sequence: Synthetic polypeptide
AKEQNPIYWARYADWLFTTPLLLLDLALLVDADEGTG

SEQ ID NO: 29 - protein, 39 aa, synthetic construct
  REGION 1..39: note = Description of Artificial Sequence: Synthetic polypeptide
AAEQNPIYWARYADWLFTTPLLLLDLALLVDADEGTKCG

SEQ ID NO: 30 - protein, 36 aa, synthetic construct
  REGION 1..36: note = Description of Artificial Sequence: Synthetic polypeptide
AKEQNPIYWARYADWLFTTPLLLLDLALLVDADECT

SEQ ID NO: 31 - protein, 37 aa, synthetic construct
  REGION 1..37: note = Description of Artificial Sequence: Synthetic polypeptide
ACEQNPIYWARYANWLFTTPLLLLNLALLVDADEGTG

SEQ ID NO: 32 - protein, 36 aa, synthetic construct
  REGION 1..36: note = Description of Artificial Sequence: Synthetic polypeptide
ACEQNPIYWARYANWLFTTPLLLLNLALLVDADEGT

SEQ ID NO: 33 - protein, 37 aa, synthetic construct
  REGION 1..37: note = Description of Artificial Sequence: Synthetic polypeptide
ACEQNPIYWARYAKWLFTTPLLLLKLALLVDADEGTG

SEQ ID NO: 34 - protein, 36 aa, synthetic construct
  REGION 1..36: note = Description of Artificial Sequence: Synthetic polypeptide
GGEQNPIYWARYADWLFTTPLLLLDLALLVNANQGT

SEQ ID NO: 35 - protein, 36 aa, synthetic construct
  REGION 1..36: note = Description of Artificial Sequence: Synthetic polypeptide
AAEQNPIYWARYADWLFTTPLLLLALALLVDADEGT

SEQ ID NO: 36 - protein, 39 aa, synthetic construct
  REGION 1..39: note = Description of Artificial Sequence: Synthetic polypeptide
AAEQNPIYWARYADWLFTTPLLLLELALLVDADEGTKCG

SEQ ID NO: 37 - protein, 36 aa, synthetic construct
  REGION 1..36: note = Description of Artificial Sequence: Synthetic polypeptide
AAEQNPIYWARYAAWLFTTPLLLLDLALLVDADEGT

SEQ ID NO: 38 - protein, 36 aa, synthetic construct
  REGION 1..36: note = Description of Artificial Sequence: Synthetic polypeptide
AAEQNPIYWARYADWLFTTALLLLDLALLVDADEGT

SEQ ID NO: 39 - protein, 36 aa, synthetic construct
  REGION 1..36: note = Description of Artificial Sequence: Synthetic polypeptide
AAEQNPIYWARYADWLFTTPLLLLELALLVDADEGT

SEQ ID NO: 40 - protein, 36 aa, synthetic construct
  REGION 1..36: note = Description of Artificial Sequence: Synthetic polypeptide
AAEQNPIYWARYAEWLFTTPLLLLDLALLVDADEGT

SEQ ID NO: 41 - protein, 39 aa, synthetic construct
  REGION 1..39: note = Description of Artificial Sequence: Synthetic polypeptide
AAEQNPIIYWARYADWLFTDLPLLLLDLLALLVDADEGT

SEQ ID NO: 42 - protein, 37 aa, synthetic construct
  REGION 1..37: note = Description of Artificial Sequence: Synthetic polypeptide
GEQNPIYWAQYADWLFTTPLLLLDLALLVDADEGTCG

SEQ ID NO: 43 - protein, 38 aa, synthetic construct
  REGION 1..38: note = Description of Artificial Sequence: Synthetic polypeptide
GGEQNPIYWARYADWLFTTPLLLDLLALLVDADEGTCG

SEQ ID NO: 44 - protein, 38 aa, synthetic construct
  REGION 1..38: note = Description of Artificial Sequence: Synthetic polypeptide
GGEQNPIYWARYADWLFTTPLLLLLDALLVDADEGTCG

SEQ ID NO: 45 - protein, 38 aa, synthetic construct
  REGION 1..38: note = Description of Artificial Sequence: Synthetic polypeptide
GGEQNPIYWARYDAWLFTTPLLLLDLALLVDADEGTCG

SEQ ID NO: 46 - protein, 38 aa, synthetic construct
  REGION 1..38: note = Description of Artificial Sequence: Synthetic polypeptide
GGEQNPIYWARYAWDLFTTPLLLLDLALLVDADEGTCG

SEQ ID NO: 47 - protein, 36 aa, synthetic construct
  REGION 1..36: note = Description of Artificial Sequence: Synthetic polypeptide
AAEQNPIYWARYADWLFTTGLLLLDLALLVDADEGT

SEQ ID NO: 48 - protein, 37 aa, synthetic construct
  REGION 1..37: note = Description of Artificial Sequence: Synthetic polypeptide
DDDEDNPIYWARYADWLFTTPLLLLHGALLVDADECT

SEQ ID NO: 49 - protein, 36 aa, synthetic construct
  REGION 1..36: note = Description of Artificial Sequence: Synthetic polypeptide
DDDEDNPIYWARYADWLFTTPLLLLHGALLVDADET

SEQ ID NO: 50 - protein, 38 aa, synthetic construct
  REGION 1..38: note = Description of Artificial Sequence: Synthetic polypeptide
DDDEDNPIYWARYAHWLFTTPLLLLHGALLVDADEGCT

SEQ ID NO: 51 - protein, 37 aa, synthetic construct
  REGION 1..37: note = Description of Artificial Sequence: Synthetic polypeptide
CDDDEDNPIYWARYAHWLFTTPLLLLHGALLVDADET

SEQ ID NO: 52 - protein, 37 aa, synthetic construct
  REGION 1..37: note = Description of Artificial Sequence: Synthetic polypeptide
DDDEDNPIYWARYAHWLFTTPLLLLHGALLVDADEGT

SEQ ID NO: 53 - protein, 37 aa, synthetic construct
  REGION 1..37: note = Description of Artificial Sequence: Synthetic polypeptide
DDDEDNPIYWARYAHWLFTTPLLLLHGALLVNADECT

SEQ ID NO: 54 - protein, 37 aa, synthetic construct
  REGION 1..37: note = Description of Artificial Sequence: Synthetic polypeptide
DDDEDNPIYWARYAHWLFTTPLLLLHGALLVNADEGT

SEQ ID NO: 55 - protein, 37 aa, synthetic construct
  REGION 1..37: note = Description of Artificial Sequence: Synthetic polypeptide
DDDEDNPIYWARYAHWLFTTPLLLLHGALLVNANECT

SEQ ID NO: 56 - protein, 37 aa, synthetic construct
  REGION 1..37: note = Description of Artificial Sequence: Synthetic polypeptide
DDDEDNPIYWARYAHWLFTTPLLLLHGALLVNANEGT

SEQ ID NO: 57 - protein, 34 aa, synthetic construct
  REGION 1..34: note = Description of Artificial Sequence: Synthetic polypeptide
AEQNPIYWARYADFLFTTPLLLLDLALLVDADET

SEQ ID NO: 58 - protein, 35 aa, synthetic construct
  REGION 1..35: note = Description of Artificial Sequence: Synthetic polypeptide
AEQNPIYFARYADWLFTTPLLLLDLALLVDADEGT

SEQ ID NO: 59 - protein, 34 aa, synthetic construct
  REGION 1..34: note = Description of Artificial Sequence: Synthetic polypeptide
AEQNPIYFARYADFLFTTPLLLLDLALLWDADET

SEQ ID NO: 60 - protein, 32 aa, synthetic construct
  REGION 1..32: note = Description of Artificial Sequence: Synthetic polypeptide
AKEDQNPYWARYADWLFTTPLLLLDLALLVDG

SEQ ID NO: 61 - protein, 32 aa, synthetic construct
  REGION 1..32: note = Description of Artificial Sequence: Synthetic polypeptide
ACEDQNPYWARYADWLFTTPLLLLDLALLVDG

SEQ ID NO: 62 - protein, 32 aa, synthetic construct
  REGION 1..32: note = Description of Artificial Sequence: Synthetic polypeptide
AEDQNPYWARYADWLFTTPLLLLDLALLVDCG

SEQ ID NO: 63 - protein, 32 aa, synthetic construct
  REGION 1..32: note = Description of Artificial Sequence: Synthetic polypeptide
AEDQNPYWARYADWLFTTPLLLLELALLVECG

SEQ ID NO: 64 - protein, 32 aa, synthetic construct
  REGION 1..32: note = Description of Artificial Sequence: Synthetic polypeptide
AKEDQNDPYWARYADWLFTTPLLLLDLALLVG

SEQ ID NO: 65 - protein, 30 aa, synthetic construct
  REGION 1..30: note = Description of Artificial Sequence: Synthetic polypeptide
AKEDQNPYWRAYADLFTPLTLLDLLALWDG

SEQ ID NO: 66 - protein, 31 aa, synthetic construct
  REGION 1..31: note = Description of Artificial Sequence: Synthetic polypeptide
AEDQNPYWARYADWLFTTPLLLLELALLVCG

SEQ ID NO: 67 - protein, 30 aa, synthetic construct
  REGION 1..30: note = Description of Artificial Sequence: Synthetic polypeptide
ACEDQNPYWRAYADLFTPLTLLDLLALWDG

SEQ ID NO: 68 - protein, 27 aa, synthetic construct
  REGION 1..27: note = Description of Artificial Sequence: Synthetic peptide
ACDDQNPWRAYLDLLFPTDTLLLDLLW

SEQ ID NO: 69 - protein, 28 aa, synthetic construct
  REGION 1..28: note = Description of Artificial Sequence: Synthetic peptide
AKDDQNPWRAYLDLLFPTDTLLLDLLWC

SEQ ID NO: 70 - protein, 27 aa, synthetic construct
  REGION 1..27: note = Description of Artificial Sequence: Synthetic peptide
ACEEQNPWRAYLELLFPTETLLLELLW

SEQ ID NO: 71 - protein, 25 aa, synthetic construct
  REGION 1..25: note = Description of Artificial Sequence: Synthetic peptide
ACDDQNPWARYLDWLFPTDTLLLDL

SEQ ID NO: 72 - protein, 24 aa, synthetic construct
  REGION 1..24: note = Description of Artificial Sequence: Synthetic peptide
CDNNNPWRAYLDLLFPTDTLLLDW

SEQ ID NO: 73 - protein, 24 aa, synthetic construct
  REGION 1..24: note = Description of Artificial Sequence: Synthetic peptide
CEEQQPWAQYLELLFPTETLLLEW

SEQ ID NO: 74 - protein, 24 aa, synthetic construct
  REGION 1..24: note = Description of Artificial Sequence: Synthetic peptide
CEEQQPWRAYLELLFPTETLLLEW

SEQ ID NO: 75 - protein, 37 aa, synthetic construct
  REGION 1..37: note = Description of Artificial Sequence: Synthetic polypeptide
CDDDDDNPNYWARYANWLFTTPLLLLNGALLVEAEET

SEQ ID NO: 76 - protein, 36 aa, synthetic construct
  REGION 1..36: note = Description of Artificial Sequence: Synthetic polypeptide
CDDDDDNPNYWARYAPWLFTTPLLLLPGALLVEAEE

SEQ ID NO: 77 - protein, 34 aa, synthetic construct
  REGION 1..34: note = Description of Artificial Sequence: Synthetic polypeptide
TEDADVLLALDLLLLPTTFLWDAYRAWYPNQECA

SEQ ID NO: 78 - protein, 20 aa, synthetic construct
  REGION 1..20: note = Description of Artificial Sequence: Synthetic peptide
AEQNPIYWARYADWLFTTPL

SEQ ID NO: 79 - protein, 21 aa, synthetic construct
  REGION 1..21: note = Description of Artificial Sequence: Synthetic peptide
AEQNPIYWARYADWLFTTPCL

SEQ ID NO: 80 - protein, 21 aa, synthetic construct
  REGION 1..21: note = Description of Artificial Sequence: Synthetic peptide
ACEQNPIYWARYADWLFTTPL

SEQ ID NO: 81 - protein, 20 aa, synthetic construct
  REGION 1..20: note = Description of Artificial Sequence: Synthetic peptide
AEQNPIYFARYADWLFTTPL

SEQ ID NO: 82 - protein, 22 aa, synthetic construct
  REGION 1..22: note = Description of Artificial Sequence: Synthetic peptide
AEQNPIYFARYADLLFPTTLAW

SEQ ID NO: 83 - protein, 22 aa, synthetic construct
  REGION 1..22: note = Description of Artificial Sequence: Synthetic peptide
AEQNPIYWARYADLLFPTTLAF

SEQ ID NO: 84 - protein, 22 aa, synthetic construct
  REGION 1..22: note = Description of Artificial Sequence: Synthetic peptide
AEQNPIYWARYADLLFPTTLAW

SEQ ID NO: 85 - protein, 21 aa, synthetic construct
  REGION 1..21: note = Description of Artificial Sequence: Synthetic peptide
KEDQNPWARYADLLFPTTLAW

SEQ ID NO: 86 - protein, 20 aa, synthetic construct
  REGION 1..20: note = Description of Artificial Sequence: Synthetic peptide
KEDQNPWARYADLLFPTTLW

SEQ ID NO: 87 - protein, 22 aa, synthetic construct
  REGION 1..22: note = Description of Artificial Sequence: Synthetic peptide
ACEDQNPWARYADLLFPTTLAW

SEQ ID NO: 88 - protein, 24 aa, synthetic construct
  REGION 1..24: note = Description of Artificial Sequence: Synthetic peptide
ACEDQNPWARYADWLFPTTLLLLD

SEQ ID NO: 89 - protein, 22 aa, synthetic construct
  REGION 1..22: note = Description of Artificial Sequence: Synthetic peptide
ACEEQNPWARYAELLFPTTLAW

SEQ ID NO: 90 - protein, 24 aa, synthetic construct
  REGION 1..24: note = Description of Artificial Sequence: Synthetic peptide
ACEEQNPWARYAEWLFPTTLLLLE

SEQ ID NO: 91 - protein, 25 aa, synthetic construct
  REGION 1..25: note = Description of Artificial Sequence: Synthetic peptide
ACEEQNPWARYLEWLFPTETLLLEL

SEQ ID NO: 92 - protein, 24 aa, synthetic construct
  REGION 1..24: note = Description of Artificial Sequence: Synthetic peptide
ACEEQNPQAEYAEWLFPTTLLLLE

SEQ ID NO: 93 - protein, 21 aa, synthetic construct
  REGION 1..21: note = Description of Artificial Sequence: Synthetic peptide
  REGION 1..21: note = See specification as filed for detailed description of substitutions and preferred embodiments
WARYADWLFTTPLLLLDLALL

SEQ ID NO: 94 - protein, 21 aa, synthetic construct
  REGION 1..21: note = Description of Artificial Sequence: Synthetic peptide
  REGION 1..21: note = See specification as filed for detailed description of substitutions and preferred embodiments
YARYADWLFTTPLLLLDLALL

SEQ ID NO: 95 - protein, 21 aa, synthetic construct
  REGION 1..21: note = Description of Artificial Sequence: Synthetic peptide
  REGION 1..21: note = See specification as filed for detailed description of substitutions and preferred embodiments
WARYSDWLFTTPLLLYDLGLL

SEQ ID NO: 96 - protein, 21 aa, synthetic construct
  REGION 1..21: note = Description of Artificial Sequence: Synthetic peptide
  REGION 1..21: note = See specification as filed for detailed description of substitutions and preferred embodiments
WARYTDWFTTPLLLYDLALLA

SEQ ID NO: 97 - protein, 21 aa, synthetic construct
  REGION 1..21: note = Description of Artificial Sequence: Synthetic peptide
  REGION 1..21: note = See specification as filed for detailed description of substitutions and preferred embodiments
WARYTDWLFTTPLLLYDLGLL

SEQ ID NO: 98 - protein, 21 aa, synthetic construct
  REGION 1..21: note = Description of Artificial Sequence: Synthetic peptide
  REGION 1..21: note = See specification as filed for detailed description of substitutions and preferred embodiments
WARYADWLFTTPLLLLDLSLL

SEQ ID NO: 99 - protein, 21 aa, synthetic construct
  REGION 1..21: note = Description of Artificial Sequence: Synthetic peptide
  REGION 1..21: note = See specification as filed for detailed description of substitutions and preferred embodiments
LLALDLLLLPTTFLWDAYRAW

SEQ ID NO: 100 - protein, 21 aa, synthetic construct
  REGION 1..21: note = Description of Artificial Sequence: Synthetic peptide
  REGION 1..21: note = See specification as filed for detailed description of substitutions and preferred embodiments
LLALDLLLLPTTFLWDAYRAY

SEQ ID NO: 101 - protein, 21 aa, synthetic construct
  REGION 1..21: note = Description of Artificial Sequence: Synthetic peptide
  REGION 1..21: note = See specification as filed for detailed description of substitutions and preferred embodiments
LLGLDYLLLPTTFLWDSYRAW

SEQ ID NO: 102 - protein, 21 aa, synthetic construct
  REGION 1..21: note = Description of Artificial Sequence: Synthetic peptide
  REGION 1..21: note = See specification as filed for detailed description of substitutions and preferred embodiments
ALLALDYLLLPTTFWDTYRAW

SEQ ID NO: 103 - protein, 21 aa, synthetic construct
  REGION 1..21: note = Description of Artificial Sequence: Synthetic peptide
  REGION 1..21: note = See specification as filed for detailed description of substitutions and preferred embodiments
LLGLDYLLLPTTFLWDTYRAW

SEQ ID NO: 104 - protein, 21 aa, synthetic construct
  REGION 1..21: note = Description of Artificial Sequence: Synthetic peptide
  REGION 1..21: note = See specification as filed for detailed description of substitutions and preferred embodiments
LLSLDLLLLPTTFLWDAYRAW

SEQ ID NO: 105 - protein, 26 aa, synthetic construct
  REGION 1..26: note = Description of Artificial Sequence: Synthetic peptide
  REGION 1..26: note = See specification as filed for detailed description of substitutions and preferred embodiments
GLAGLLGLEGLLGLPLGLLEGLWLGL

SEQ ID NO: 106 - protein, 26 aa, synthetic construct
  REGION 1..26: note = Description of Artificial Sequence: Synthetic peptide
  REGION 1..26: note = See specification as filed for detailed description of substitutions and preferred embodiments
LGLWLGELLGLPLGLLGELGLLGALG

SEQ ID NO: 107 - protein, 20 aa, synthetic construct
  REGION 1..20: note = Description of Artificial Sequence: Synthetic peptide
  REGION 1..20: note = See specification as filed for detailed description of substitutions and preferred embodiments
WRAYLDLLFPTDTLLLDLLW

SEQ ID NO: 108 - protein, 20 aa, synthetic construct
  REGION 1..20: note = Description of Artificial Sequence: Synthetic peptide
  REGION 1..20: note = See specification as filed for detailed description of substitutions and preferred embodiments
WLLDLLLTDTPFLLDLYARW

SEQ ID NO: 109 - protein, 18 aa, synthetic construct
  REGION 1..18: note = Description of Artificial Sequence: Synthetic peptide
  REGION 1..18: note = See specification as filed for detailed description of substitutions and preferred embodiments
WARYLEWLFPTETLLLEL

SEQ ID NO: 110 - protein, 18 aa, synthetic construct
  REGION 1..18: note = Description of Artificial Sequence: Synthetic peptide
  REGION 1..18: note = See specification as filed for detailed description of substitutions and preferred embodiments
WAQYLELLFPTETLLLEW

SEQ ID NO: 111 - protein, 18 aa, synthetic construct
  REGION 1..18: note = Description of Artificial Sequence: Synthetic peptide
  REGION 1..18: note = See specification as filed for detailed description of substitutions and preferred embodiments
LELLLTETPFLWELYRAW

SEQ ID NO: 112 - protein, 18 aa, synthetic construct
  REGION 1..18: note = Description of Artificial Sequence: Synthetic peptide
  REGION 1..18: note = See specification as filed for detailed description of substitutions and preferred embodiments
WELLLTETPFLLELYQAW

SEQ ID NO: 113 - protein, 17 aa, synthetic construct
  REGION 1..17: note = Description of Artificial Sequence: Synthetic peptide
  REGION 1..17: note = See specification as filed for detailed description of substitutions and preferred embodiments
WLFTTPLLLLNGALLVE

SEQ ID NO: 114 - protein, 17 aa, synthetic construct
  REGION 1..17: note = Description of Artificial Sequence: Synthetic peptide
  REGION 1..17: note = See specification as filed for detailed description of substitutions and preferred embodiments
WLFTTPLLLLPGALLVE

SEQ ID NO: 115 - protein, 15 aa, synthetic construct
  REGION 1..15: note = Description of Artificial Sequence: Synthetic peptide
  REGION 1..15: note = See specification as filed for detailed description of substitutions and preferred embodiments
WARYADLLFPTTLAW

SEQ ID NO: 116 - protein, 17 aa, synthetic construct
  REGION 1..17: note = Description of Artificial Sequence: Synthetic peptide
  REGION 1..17: note = See specification as filed for detailed description of substitutions and preferred embodiments
EVLLAGNLLLLPTTFLW

SEQ ID NO: 117 - protein, 17 aa, synthetic construct
  REGION 1..17: note = Description of Artificial Sequence: Synthetic peptide
  REGION 1..17: note = See specification as filed for detailed description of substitutions and preferred embodiments
EVLLAGPLLLLPTTFLW

SEQ ID NO: 118 - protein, 15 aa, synthetic construct
  REGION 1..15: note = Description of Artificial Sequence: Synthetic peptide
  REGION 1..15: note = See specification as filed for detailed description of substitutions and preferred embodiments
WALTTPFLLDAYRAW

SEQ ID NO: 119 - protein, 24 aa, synthetic construct
  REGION 1..24: note = Description of Artificial Sequence: Synthetic peptide
  REGION 1..24: note = See specification as filed for detailed description of substitutions and preferred embodiments
NLEGFFATLGGEIALWSLVVLAIE

SEQ ID NO: 120 - protein, 22 aa, synthetic construct
  REGION 1..22: note = Description of Artificial Sequence: Synthetic peptide
  REGION 1..22: note = See specification as filed for detailed description of substitutions and preferred embodiments
EGFFATLGGEIALWSDVVLAIE

SEQ ID NO: 121 - protein, 22 aa, synthetic construct
  REGION 1..22: note = Description of Artificial Sequence: Synthetic peptide
  REGION 1..22: note = See specification as filed for detailed description of substitutions and preferred embodiments
EGFFATLGGEIPLWSDVVLAIE

SEQ ID NO: 122 - protein, 24 aa, synthetic construct
  REGION 1..24: note = Description of Artificial Sequence: Synthetic peptide
  REGION 1..24: note = See specification as filed for detailed description of substitutions and preferred embodiments
EIALVVLSWLAIEGGLTAFFGELN

SEQ ID NO: 123 - protein, 22 aa, synthetic construct
  REGION 1..22: note = Description of Artificial Sequence: Synthetic peptide
  REGION 1..22: note = See specification as filed for detailed description of substitutions and preferred embodiments
EIALVVDSWLAIEGGLTAFFGE

SEQ ID NO: 124 - protein, 22 aa, synthetic construct
  REGION 1..22: note = Description of Artificial Sequence: Synthetic peptide
  REGION 1..22: note = See specification as filed for detailed description of substitutions and preferred embodiments
EIALVVDSWLPIEGGLTAFFGE

SEQ ID NO: 125 - protein, 21 aa, synthetic construct
  REGION 1..21: note = Description of Artificial Sequence: Synthetic peptide
  REGION 1..21: note = See specification as filed for detailed description of substitutions and preferred embodiments
ILDLVFGLLFAVTSVDFLVQW

SEQ ID NO: 126 - protein, 21 aa, synthetic construct
  REGION 1..21: note = Description of Artificial Sequence: Synthetic peptide
  REGION 1..21: note = See specification as filed for detailed description of substitutions and preferred embodiments
WQVLFDVSTVAFLLGFVLDLI

SEQ ID NO: 127 - protein, 34 aa, synthetic construct
  REGION 1..34: note = Description of Artificial Sequence: Synthetic polypeptide
AEQNPIYWARYADWLFTTPLLLLDLALLVDADET

SEQ ID NO: 128 - protein, 34 aa, synthetic construct
  REGION 1..34: note = Description of Artificial Sequence: Synthetic polypeptide
AEQNPIYWARYAEWLFTTPLLLLELALLVEAEET

SEQ ID NO: 129 - protein, 33 aa, synthetic construct
  REGION 1..33: note = Description of Artificial Sequence: Synthetic polypeptide
ADDQNPWRAYLDLLFPDTTDLLLLDLLWDADET

SEQ ID NO: 130 - protein, 33 aa, synthetic construct
  REGION 1..33: note = Description of Artificial Sequence: Synthetic polypeptide
AEEQNPWRAYLELLFPETTELLLLELLWEAEET

SEQ ID NO: 131 - protein, 34 aa, synthetic construct
  REGION 1..34: note = Description of Artificial Sequence: Synthetic polypeptide
  MOD_RES 13: note = Gla
AEQNPIYWARYAXWLFTTPLLLLDLALLVDADET

SEQ ID NO: 132 - protein, 34 aa, synthetic construct
  REGION 1..34: note = Description of Artificial Sequence: Synthetic polypeptide
  MOD_RES 24: note = Gla
AEQNPIYWARYADWLFTTPLLLLXLALLVDADET

SEQ ID NO: 133 - protein, 34 aa, synthetic construct
  REGION 1..34: note = Description of Artificial Sequence: Synthetic polypeptide
  MOD_RES 13: note = Gla
  MOD_RES 24: note = Gla
AEQNPIYWARYAXWLFTTPLLLLXLALLVDADET

SEQ ID NO: 134 - protein, 34 aa, synthetic construct; identical to SEQ ID NO: 131
  REGION 1..34: note = Description of Artificial Sequence: Synthetic polypeptide
  MOD_RES 13: note = Aad

SEQ ID NO: 135 - protein, 34 aa, synthetic construct; identical to SEQ ID NO: 132
  REGION 1..34: note = Description of Artificial Sequence: Synthetic polypeptide
  MOD_RES 24: note = Aad

SEQ ID NO: 136 - protein, 34 aa, synthetic construct; identical to SEQ ID NO: 133
  REGION 1..34: note = Description of Artificial Sequence: Synthetic polypeptide
  MOD_RES 13: note = Aad
  MOD_RES 24: note = Aad

SEQ ID NO: 137 - protein, 34 aa, synthetic construct; identical to SEQ ID NO: 133
  REGION 1..34: note = Description of Artificial Sequence: Synthetic polypeptide
  MOD_RES 13: note = Gla
  MOD_RES 24: note = Aad

SEQ ID NO: 138 - protein, 34 aa, synthetic construct; identical to SEQ ID NO: 133
  REGION 1..34: note = Description of Artificial Sequence: Synthetic polypeptide
  MOD_RES 13: note = Aad
  MOD_RES 24: note = Gla

SEQ ID NO: 139 - protein, 36 aa, synthetic construct; identical to SEQ ID NO: 14
  REGION 1..36: note = Description of Artificial Sequence: Synthetic polypeptide

SEQ ID NO: 140 - protein, 36 aa, synthetic construct; identical to SEQ ID NO: 14
  REGION 1..36: note = Description of Artificial Sequence: Synthetic polypeptide

SEQ ID NO: 141 - protein, 36 aa, synthetic construct
  REGION 1..36: note = Description of Artificial Sequence: Synthetic polypeptide
AAEQNPIYWARYADWLFTTPLLLLDLALLVDADEGT

SEQ ID NO: 142 - protein, 35 aa, synthetic construct; identical to SEQ ID NO: 7
  REGION 1..35: note = Description of Artificial Sequence: Synthetic polypeptide

SEQ ID NO: 143 - protein, 35 aa, synthetic construct
  REGION 1..35: note = Description of Artificial Sequence: Synthetic polypeptide
AEQNPIYWARYANWLFTTPLLLLNLALLVDADEGT

SEQ ID NO: 144 - protein, 35 aa, synthetic construct
  REGION 1..35: note = Description of Artificial Sequence: Synthetic polypeptide
AEQNPIYWARYAKWLFTTPLLLLKLALLVDADEGT

SEQ ID NO: 145 - protein, 36 aa, synthetic construct; identical to SEQ ID NO: 34
  REGION 1..36: note = Description of Artificial Sequence: Synthetic polypeptide

SEQ ID NO: 146 - protein, 36 aa, synthetic construct; identical to SEQ ID NO: 35
  REGION 1..36: note = Description of Artificial Sequence: Synthetic polypeptide

SEQ ID NO: 147 - protein, 36 aa, synthetic construct; identical to SEQ ID NO: 37
  REGION 1..36: note = Description of Artificial Sequence: Synthetic polypeptide

SEQ ID NO: 148 - protein, 36 aa, synthetic construct; identical to SEQ ID NO: 38
  REGION 1..36: note = Description of Artificial Sequence: Synthetic polypeptide

SEQ ID NO: 149 - protein, 36 aa, synthetic construct; identical to SEQ ID NO: 39
  REGION 1..36: note = Description of Artificial Sequence: Synthetic polypeptide

SEQ ID NO: 150 - protein, 36 aa, synthetic construct; identical to SEQ ID NO: 40
  REGION 1..36: note = Description of Artificial Sequence: Synthetic polypeptide

SEQ ID NO: 151 - protein, 39 aa, synthetic construct; identical to SEQ ID NO: 41
  REGION 1..39: note = Description of Artificial Sequence: Synthetic polypeptide

SEQ ID NO: 152 - protein, 34 aa, synthetic construct
  REGION 1..34: note = Description of Artificial Sequence: Synthetic polypeptide
GEQNPIYWAQYADWLFTTPLLLLDLALLVDADEG

SEQ ID NO: 153 - protein, 35 aa, synthetic construct
  REGION 1..35: note = Description of Artificial Sequence: Synthetic polypeptide
GGEQNPIYWARYADWLFTTPLLLDLLALLVDADEG

SEQ ID NO: 154 - protein, 35 aa, synthetic construct
  REGION 1..35: note = Description of Artificial Sequence: Synthetic polypeptide
GGEQNPIYWARYADWLFTTPLLLLLDALLVDADEG

SEQ ID NO: 155 - protein, 36 aa, synthetic construct
  REGION 1..36: note = Description of Artificial Sequence: Synthetic polypeptide
GGEQNPIYWARYDAWLFTTPLLLLDLALLVDADEGT

SEQ ID NO: 156 - protein, 35 aa, synthetic construct
  REGION 1..35: note = Description of Artificial Sequence: Synthetic polypeptide
GGEQNPIYWARYAWDLFTTPLLLLDLALLVDADEG

SEQ ID NO: 157 - protein, 36 aa, synthetic construct; identical to SEQ ID NO: 47
  REGION 1..36: note = Description of Artificial Sequence: Synthetic polypeptide

SEQ ID NO: 158 - protein, 34 aa, synthetic construct
  REGION 1..34: note = Description of Artificial Sequence: Synthetic polypeptide
DDDEDNPIYWARYADWLFTTPLLLLHGALLVDAD

SEQ ID NO: 159 - protein, 35 aa, synthetic construct
  REGION 1..35: note = Description of Artificial Sequence: Synthetic polypeptide
DDDEDNPIYWARYAHWLFTTPLLLLHGALLVDADE

SEQ ID NO: 160 - protein, 33 aa, synthetic construct
  REGION 1..33: note = Description of Artificial Sequence: Synthetic polypeptide
CEQNPIYWARYADWHFTTPLLLLDLALLVDADE

SEQ ID NO: 161 - protein, 34 aa, synthetic construct; identical to SEQ ID NO: 57
  REGION 1..34: note = Description of Artificial Sequence: Synthetic polypeptide

SEQ ID NO: 162 - protein, 33 aa, synthetic construct
  REGION 1..33: note = Description of Artificial Sequence: Synthetic polypeptide
AEQNPIYFARYADWLFTTPLLLLDLALLVDADE

SEQ ID NO: 163 - protein, 34 aa, synthetic construct; identical to SEQ ID NO: 59
  REGION 1..34: note = Description of Artificial Sequence: Synthetic polypeptide

SEQ ID NO: 164 - protein, 32 aa, synthetic construct
  REGION 1..32: note = Description of Artificial Sequence: Synthetic polypeptide
ADNNPWIYARYADLTTFPLLLLDLALLVDFDD

SEQ ID NO: 165 - protein, 32 aa, synthetic construct
  REGION 1..32: note = Description of Artificial Sequence: Synthetic polypeptide
ADNNPFIYARYADLTTWPLLLLDLALLVDFDD

SEQ ID NO: 166 - protein, 32 aa, synthetic construct
  REGION 1..32: note = Description of Artificial Sequence: Synthetic polypeptide
ADNNPFIYARYADLTTFPLLLLDLALLVDWDD

SEQ ID NO: 167 - protein, 32 aa, synthetic construct
  REGION 1..32: note = Description of Artificial Sequence: Synthetic polypeptide
ADNNPFPYARYADLTTWILLLLDLALLVDFDD

SEQ ID NO: 168 - protein, 32 aa, synthetic construct
  REGION 1..32: note = Description of Artificial Sequence: Synthetic polypeptide
ADNNPFIYAYRADLTTFPLLLLDLALLVDWDD

SEQ ID NO: 169 - protein, 32 aa, synthetic construct
  REGION 1..32: note = Description of Artificial Sequence: Synthetic polypeptide
ADNNPFIYATYADLRTFPLLLLDLALLVDWDD

SEQ ID NO: 170 - protein, 33 aa, synthetic construct
  REGION 1..33: note = Description of Artificial Sequence: Synthetic polypeptide
TEDADVLLALDLLLLPTTFLWDAYRAWYPNQEA

SEQ ID NO: 171 - protein, 30 aa, synthetic construct
  REGION 1..30: note = Description of Artificial Sequence: Synthetic polypeptide
AEDQNPYWARYADWLFTTPLLLLDLALLVD

SEQ ID NO: 172 - protein, 30 aa, synthetic construct
  REGION 1..30: note = Description of Artificial Sequence: Synthetic polypeptide
AEDQNPYWARYADWLFTTPLLLLELALLVE

SEQ ID NO: 173 - protein, 28 aa, synthetic construct
  REGION 1..28: note = Description of Artificial Sequence: Synthetic peptide
AEDQNPYWRAYADLFTPLTLLDLLALWD

SEQ ID NO: 174 - protein, 26 aa, synthetic construct
  REGION 1..26: note = Description of Artificial Sequence: Synthetic peptide
ADDQNPWRAYLDLLFPTDTLLLDLLW

SEQ ID NO: 175 - protein, 30 aa, synthetic construct
  REGION 1..30: note = Description of Artificial Sequence: Synthetic polypeptide
ADDQNPWRAYLDLLFPTDTLLLDLLWDADE

SEQ ID NO: 176 - protein, 26 aa, synthetic construct
  REGION 1..26: note = Description of Artificial Sequence: Synthetic peptide
  MOD_RES 12: note = Gla
ADDQNPWRAYLXLLFPTDTLLLDLLW

SEQ ID NO: 177 - protein, 26 aa, synthetic construct
  REGION 1..26: note = Description of Artificial Sequence: Synthetic peptide
  MOD_RES 18: note = Gla
ADDQNPWRAYLDLLFPTXTLLLDLLW

SEQ ID NO: 178 - protein, 26 aa, synthetic construct
  REGION 1..26: note = Description of Artificial Sequence: Synthetic peptide
  MOD_RES 23: note = Gla
ADDQNPWRAYLDLLFPTDTLLLXLLW

SEQ ID NO: 179 - protein, 26 aa, synthetic construct
  REGION 1..26: note = Description of Artificial Sequence: Synthetic peptide
  MOD_RES 12: note = Gla
  MOD_RES 18: note = Gla
ADDQNPWRAYLXLLFPTXTLLLDLLW

SEQ ID NO: 180 - protein, 26 aa, synthetic construct
  REGION 1..26: note = Description of Artificial Sequence: Synthetic peptide
  MOD_RES 12: note = Gla
  MOD_RES 23: note = Gla
ADDQNPWRAYLXLLFPTDTLLLXLLW

SEQ ID NO: 181 - protein, 26 aa, synthetic construct
  REGION 1..26: note = Description of Artificial Sequence: Synthetic peptide
  MOD_RES 18: note = Gla
  MOD_RES 23: note = Gla
ADDQNPWRAYLDLLFPTXTLLLXLLW

SEQ ID NO: 182 - protein, 26 aa, synthetic construct
  REGION 1..26: note = Description of Artificial Sequence: Synthetic peptide
  MOD_RES 12: note = Gla
  MOD_RES 18: note = Gla
  MOD_RES 23: note = Gla
ADDQNPWRAYLXLLFPTXTLLLXLLW

SEQ ID NO: 183 - protein, 26 aa, synthetic construct
  REGION 1..26: note = Description of Artificial Sequence: Synthetic peptide
  MOD_RES 12: note = Aad
ADDQNPWRAYLXLLFPTDTLLLDLLW

SEQ ID NO: 184 - protein, 26 aa, synthetic construct
  REGION 1..26: note = Description of Artificial Sequence: Synthetic peptide
  MOD_RES 18: note = Aad
ADDQNPWRAYLDLLFPTXTLLLDLLW

SEQ ID NO: 185 - protein, 26 aa, synthetic construct
  REGION 1..26: note = Description of Artificial Sequence: Synthetic peptide
  MOD_RES 23: note = Aad
ADDQNPWRAYLDLLFPTDTLLLXLLW

SEQ ID NO: 186 - protein, 26 aa, synthetic construct
  REGION 1..26: note = Description of Artificial Sequence: Synthetic peptide
  MOD_RES 12: note = Aad
  MOD_RES 18: note = Aad
ADDQNPWRAYLXLLFPTXTLLLDLLW

SEQ ID NO: 187 - protein, 26 aa, synthetic construct
  REGION 1..26: note = Description of Artificial Sequence: Synthetic peptide
  MOD_RES 12: note = Aad
  MOD_RES 23: note = Aad
ADDQNPWRAYLXLLFPTDTLLLXLLW

SEQ ID NO: 188 - protein, 26 aa, synthetic construct
  REGION 1..26: note = Description of Artificial Sequence: Synthetic peptide
  MOD_RES 18: note = Aad
  MOD_RES 23: note = Aad
ADDQNPWRAYLDLLFPTXTLLLXLLW

SEQ ID NO: 189 - protein, 26 aa, synthetic construct
  REGION 1..26: note = Description of Artificial Sequence: Synthetic peptide
  MOD_RES 12: note = Aad
  MOD_RES 18: note = Aad
  MOD_RES 23: note = Aad
ADDQNPWRAYLXLLFPTXTLLLXLLW

SEQ ID NO: 190 - protein, 26 aa, synthetic construct
  REGION 1..26: note = Description of Artificial Sequence: Synthetic peptide
  MOD_RES 12: note = Gla
  MOD_RES 18: note = Aad
ADDQNPWRAYLXLLFPTXTLLLDLLW

SEQ ID NO: 191 - protein, 26 aa, synthetic construct
  REGION 1..26: note = Description of Artificial Sequence: Synthetic peptide
  MOD_RES 12: note = Gla
  MOD_RES 23: note = Aad
ADDQNPWRAYLXLLFPTDTLLLXLLW

SEQ ID NO: 192 - protein, 26 aa, synthetic construct
  REGION 1..26: note = Description of Artificial Sequence: Synthetic peptide
  MOD_RES 12: note = Gla
  MOD_RES 18: note = Gla
  MOD_RES 23: note = Aad
ADDQNPWRAYLXLLFPTXTLLLXLLW

SEQ ID NO: 193 - protein, 26 aa, synthetic construct
  REGION 1..26: note = Description of Artificial Sequence: Synthetic peptide
  MOD_RES 12: note = Aad
  MOD_RES 18: note = Gla
ADDQNPWRAYLXLLFPTXTLLLDLLW

SEQ ID NO: 194 - protein, 26 aa, synthetic construct
  REGION 1..26: note = Description of Artificial Sequence: Synthetic peptide
  MOD_RES 12: note = Aad
  MOD_RES 23: note = Gla
ADDQNPWRAYLXLLFPTDTLLLXLLW

SEQ ID NO: 195 - protein, 26 aa, synthetic construct
  REGION 1..26: note = Description of Artificial Sequence: Synthetic peptide
  MOD_RES 12: note = Gla
  MOD_RES 18: note = Aad
  MOD_RES 23: note = Gla
ADDQNPWRAYLXLLFPTXTLLLXLLW

SEQ ID NO: 196 - protein, 29 aa, synthetic construct
  REGION 1..29: note = Description of Artificial Sequence: Synthetic peptide
GEEQNPWLGAYLDLLFPLELLGLLELGLW

SEQ ID NO: 197 - protein, 28 aa, synthetic construct
  REGION 1..28: note = Description of Artificial Sequence: Synthetic peptide
ADDDDDDPWQAYLDLLFPTDTLLLDLLW

SEQ ID NO: 198 - protein, 26 aa, synthetic construct
  REGION 1..26: note = Description of Artificial Sequence: Synthetic peptide
AEEQNPWRAYLELLFPTETLLLELLW

SEQ ID NO: 199 - protein, 24 aa, synthetic construct
  REGION 1..24: note = Description of Artificial Sequence: Synthetic peptide
ADDQNPWARYLDWLFPTDTLLLDL

SEQ ID NO: 200 - protein, 23 aa, synthetic construct
  REGION 1..23: note = Description of Artificial Sequence: Synthetic peptide
DNNNPWRAYLDLLFPTDTLLLDW

SEQ ID NO: 201 - protein, 24 aa, synthetic construct
  REGION 1..24: note = Description of Artificial Sequence: Synthetic peptide
AEEQNPWARYLEWLFPTETLLLEL

SEQ ID NO: 202 - protein, 25 aa, synthetic construct
  REGION 1..25: note = Description of Artificial Sequence: Synthetic peptide
DDDDDDPWQAYLDLFPTDTLALDLW

SEQ ID NO: 203 - protein, 23 aa, synthetic construct
  REGION 1..23: note = Description of Artificial Sequence: Synthetic peptide
EEQQPWAQYLELLFPTETLLLEW

SEQ ID NO: 204 - protein, 23 aa, synthetic construct
  REGION 1..23: note = Description of Artificial Sequence: Synthetic peptide
EEQQPWRAYLELLFPTETLLLEW

SEQ ID NO: 205 - protein, 23 aa, synthetic construct
  REGION 1..23: note = Description of Artificial Sequence: Synthetic peptide
AEDQNPWARYADWLFPTTLLLLD

SEQ ID NO: 206 - protein, 23 aa, synthetic construct
  REGION 1..23: note = Description of Artificial Sequence: Synthetic peptide
AEEQNPWARYAEWLFPTTLLLLE

SEQ ID NO: 207 - protein, 21 aa, synthetic construct
  REGION 1..21: note = Description of Artificial Sequence: Synthetic peptide
AEDQNPWARYADLLFPTTLAW

SEQ ID NO: 208 - protein, 21 aa, synthetic construct
  REGION 1..21: note = Description of Artificial Sequence: Synthetic peptide
AEEQNPWARYAELLFPTTLAW

SEQ ID NO: 209 - protein, 36 aa, synthetic construct
  REGION 1..36: note = Description of Artificial Sequence: Synthetic polypeptide
DDDDDNPNYWARYANWLFTTPLLLLNGALLVEAEET

SEQ ID NO: 210 - protein, 36 aa, synthetic construct
  REGION 1..36: note = Description of Artificial Sequence: Synthetic polypeptide
DDDDDNPNYWARYAPWLFTTPLLLLPGALLVEAEET

SEQ ID NO: 211 - protein, 34 aa, synthetic construct; identical to SEQ ID NO: 59
  REGION 1..34: note = Description of Artificial Sequence: Synthetic polypeptide

SEQ ID NO: 212 - protein, 34 aa, synthetic construct; identical to SEQ ID NO: 57
  REGION 1..34: note = Description of Artificial Sequence: Synthetic polypeptide

SEQ ID NO: 213 - protein, 20 aa, synthetic construct
  REGION 1..20: note = Description of Artificial Sequence: Synthetic peptide
AEQNPIYWARYADWLFTTPL

SEQ ID NO: 214 - protein, 22 aa, synthetic construct
  REGION 1..22: note = Description of Artificial Sequence: Synthetic peptide
AEQNPIYFARYADLLFPTTLAW

SEQ ID NO: 215 - protein, 22 aa, synthetic construct
  REGION 1..22: note = Description of Artificial Sequence: Synthetic peptide
AEQNPIYWARYADLLFPTTLAF

SEQ ID NO: 216 - protein, 22 aa, synthetic construct
  REGION 1..22: note = Description of Artificial Sequence: Synthetic peptide
AEQNPIYWARYADLLFPTTLAW

SEQ ID NO: 217 - protein, 20 aa, synthetic construct
  REGION 1..20: note = Description of Artificial Sequence: Synthetic peptide
AEQNPIYFARYADWLFTTPL

SEQ ID NO: 218 - protein, 20 aa, synthetic construct
  REGION 1..20: note = Description of Artificial Sequence: Synthetic peptide
EDQNPWARYADLLFPTTLAW

SEQ ID NO: 219 - protein, 34 aa, synthetic construct
  REGION 1..34: note = Description of Artificial Sequence: Synthetic polypeptide
GLAGLAGLLGLEGLLGLPLGLLEGLWLGLELEGN

SEQ ID NO: 220 - protein, 32 aa, synthetic construct
  REGION 1..32: note = Description of Artificial Sequence: Synthetic polypeptide
EQNPIYILDLVFGLLFAVTSVDFLVQWDDAGD

SEQ ID NO: 221 - protein, 24 aa, synthetic construct
  REGION 1..24: note = Description of Artificial Sequence: Synthetic peptide
NLEGFFATLGGEIALWSLVVLAIE

SEQ ID NO: 222 - protein, 24 aa, synthetic construct
  REGION 1..24: note = Description of Artificial Sequence: Synthetic peptide
NNEGFFATLGGEIALWSDVVLAIE

SEQ ID NO: 223 - protein, 25 aa, synthetic construct
  REGION 1..25: note = Description of Artificial Sequence: Synthetic peptide
DNNEGFFATLGGEIPLWSDVVLAIE